(19) United States
(12) Patent Application Publication
(10) Pub. No.: US 2018/0349548 A1
(43) Pub. Date: Dec. 6, 2018

(54) METHODS AND COMPOSITIONS THAT UTILIZE TRANSCRIPTOME SEQUENCING DATA IN MACHINE LEARNING-BASED CLASSIFICATION

(71) Applicant: VERACYTE, INC., South San Francisco, CA (US)

(72) Inventors: P. Sean Walsh, South San Francisco, CA (US); Giulia C. Kennedy, San Francisco, CA (US); Kevin Travers, South San Francisco, CA (US); Zhanzhi Hu, South San Francisco, CA (US); Su Yeon Kim, South San Francisco, CA (US); Jing Huang, South San Francisco, CA (US)

(21) Appl. No.: 15/934,666

(22) Filed: Mar. 23, 2018

Related U.S. Application Data
(63) Continuation of application No. PCT/US2016/053578, filed on Sep. 23, 2016.
(60) Provisional application No. 62/233,207, filed on Sep. 25, 2015.

(51) Int. Cl. G06F 19/12; G06F 19/20; G06F 19/24; G06F 19/26; G06F 17/18; G16H 50/20

(52) U.S. Cl. CPC G06F 19/12; G06F 19/20; G06F 19/24; G06N 3/02; G06F 17/18; G16H 50/20; G06F 19/26

(57) ABSTRACT

Provided herein are methods and systems for producing a modified biological dataset by flagging or removing a nucleic acid sequence from the biological dataset that is assigned a noise-call to produce the modified biological dataset. The noise-call may be based on comparing a gene expression level, sequence information, or a combination thereof with a nucleic acid sequence of a control sample.

### CROSS REFERENCE

This application claims priority to U.S. Provisional Patent Application Ser. No. 62/233,207, filed Sep. 25, 2015, which is entirely incorporated herein by reference.

### BACKGROUND

Massively parallel next generation sequencing of RNA (RNASeq) has revolutionized the way transcriptome messages are detected, decoded, interpreted, and utilized. RNASeq data is multidimensional reporting 1) mRNA expression levels, a quantitative measure, and 2) sequence information, a categorical determination (theoretically non-quantitative) of the actual sequences contained within a specific region of the genome (for example at any given position the nucleotide sequence may be adenine, cytosine, guanine, or thymine). The power of RNASeq lies in being able to use expression levels and sequence information (e.g., variants) simultaneously. However, combining datasets generated at different times or by different laboratories and utilizing these datasets in machine learning applications presents a challenge.

### SUMMARY

An aspect of the present disclosure provides a method for processing a biological sample. The method may comprise (a) assaying one or more nucleic acid sequences from the biological sample to obtain a biological dataset comprising gene expression levels, sequence variant information, or a combination thereof corresponding to the one or more nucleic acid sequences; (b) comparing the biological dataset assayed in (a) to a second dataset comprising gene expression levels, sequence variant information, or a combination thereof corresponding to one or more nucleic acid sequences of a control sample; (c) assigning a call to the one or more nucleic acid sequences of the biological dataset based on the comparing of (b), wherein the call is a no-call, a reference-call, or a noise-call; (d) assigning the noise-call to a nucleic acid sequence of the biological dataset; and (e) upon assigning the noise-call to the nucleic acid sequence, (i) flagging the nucleic acid sequence within the biological dataset, or (ii) removing the nucleic acid sequence from the biological dataset, to produce the modified biological dataset.

In some embodiments, the biological dataset comprises the gene expression levels. In some embodiments, the biological dataset comprises the sequence variant information. In some embodiments, the second dataset comprises the gene expression levels. In some embodiments, the second dataset comprises the sequence variant information.

In some embodiments, the assigning further comprises applying (i) a DESeq Wald-test, a Limma test, a Fisher's extract test, or any combination thereof, (ii) a Hierarchical Ordered Partitioning And Collapsing Hybrid (HOPACH) cluster, or (iii) a combination thereof to the biological dataset. In some embodiments, the assigning further comprises applying the HOPACH cluster. In some embodiments, the assigning further comprises applying the Limma test and the DESeq Wald-test.

In some embodiments, the flagging comprises weighting the nucleic acid sequence differently from nucleic acid sequences of the biological dataset that is not assigned the noise-call. In some embodiments, the assaying comprises assaying a first portion of the biological sample separately from assaying a second portion of the biological sample. In some embodiments, the first portion is assayed at a different time, by a different operator, employing a different equipment type, employing a different reagent, or any combination thereof compared to the second portion. In some embodiments, the biological sample is obtained from a first source and a second source, wherein the first source and the second source is different.

In some embodiments, the comparing further comprises determining a difference in expression level between the gene expression levels of the one or more nucleic acid sequences from the biological sample compared to the gene expression levels of one or more nucleic acid sequences of the control sample having at least about 90% homology to the one or more nucleic acid sequences from the biological sample. In some embodiments, when the difference in expression level for a given nucleic acid sequence of the biological sample is greater than about 10%, then assigning the noise-call to the given nucleic acid sequence in (c).

In some embodiments, the comparing further comprises determining a presence or an absence of a fusion in the one or more nucleic acid sequences of the biological sample compared to a presence or an absence of the fusion in one or more nucleic acid sequences of the control sample having at least about 90% homology to the one or more nucleic acid sequences of the biological sample. In some embodiments, when the fusion is present in a given nucleic acid sequence of the biological sample and is not present in a nucleic acid sequence of the control sample, then assigning the noise-call to the given nucleic acid sequence in (c). In some embodiments, the comparing employs a fusion panel that comprises less than about 200 fusions.

In some embodiments, the comparing further comprises determining a presence or an absence of a sequence variant, a sequence variant count number, or a combination thereof in the one or more nucleic acid sequences of the biological sample compared to a present or an absence of the sequence variant, the sequence variant count number, or a combination thereof in one or more nucleic acid sequences of the control sample having at least about 90% homology to the one or more nucleic acid sequences of the biological sample. In some embodiments, when the sequence variant is present in a given nucleic acid sequence of the biological sample and is not present in a nucleic acid sequence of the control sample, then assigning the noise-call to the given nucleic acid sequence in (c). In some embodiments, when a raw count, a normalized count, or a number of counts of the sequence variant of a given nucleic acid sequence of the biological sample differs from a raw count, a normalized count or a number of counts of a sequence variant of a nucleic acid sequence in the control sample having at least about 90% homology to the given nucleic acid sequence of the biological sample, then assigning the noise-call to the given nucleic acid sequence in (c). In some embodiments, the comparing employs a sequence variant panel that comprises less than about 900 sequence variants.

In some embodiments, the biological sample is independent from the control sample. In some embodiments, the gene expression levels assayed in (b) are measured by microarray, SAGE, blotting, RT-PCR, sequencing, and/or quantitative PCR. In some embodiments, the assaying comprises next generation sequencing of RNA (RNASeq).

In some embodiments, the sequence variants comprises a polymorphism, a mutation, a fusion, a splice variant, a copy number alteration, or any combination thereof. In some embodiments, nucleic sequence assigned the noise-call in (d) comprises a transcript degradation, an impartial fragmentation, an incomplete library preparation, a 3′ to 5′ bias, a polymerase processivity, a polymerase sequence bias, or any combination thereof. In some embodiments, the modified biological dataset comprises one or more nucleic acid sequences assigned a no-call, a reference call, or a combination thereof. In some embodiments, at least about 70% of the one or more nucleic acid sequences of the biological sample has at least about 90% sequence homology to a nucleic acid sequence of the one or more nucleic acid sequences of the control sample.

In some embodiments, the control sample comprises a housekeeping gene. In some embodiments, the one or more nucleic acid sequences assayed in (a) is less than about 10 nucleic acid sequences. In some embodiments, the one or more nucleic acid sequences of the control sample comprises less than about 900 nucleic acid sequences.

In some embodiments, the nucleic acid sequence assigned the noise-call in (d) comprises at least about 90% sequence homology to BRAF, HRAS, KRAS, NRAS, TSHR, or RET, or any fragment thereof. In some embodiments, the nucleic acid sequence assigned the noise-call in (d) comprises at least about 90% sequence homology to TSHR, RET, NRAS, TP53, PAX8, FAT1, VT11A, BRAF, HRAS, or KRAS, or any fragment thereof.

In some embodiments, the method further comprises inputting the modified biological dataset into a trained algorithm. In some embodiments, the method further comprises employing the modified biological dataset to train a trained algorithm. In some embodiments, the trained algorithm employs a Support Vector Machine (SVM) model, a Random Forest (RF) model, a Least Absolute Shrinkage and Selection Operator (LASSO) model, an Ensemble 1 model, a Penalized Logistic Regression (PLR) model, a Classification And Regression Trees (CART) model, or any combination thereof. In some embodiments, the trained algorithm classifies the biological sample as negative or positive for a disease. In some embodiments, the disease is cancer. In some embodiments, the cancer is thyroid cancer. In some embodiments, the biological sample is classified as negative for the disease at an accuracy of at least about 80%. In some embodiments, the biological sample is classified as negative for the disease at a specificity of at least about 70%. In some embodiments, the biological sample is classified as negative for the disease at a sensitivity of at least about 70%. In some embodiments, the biological sample is classified as having the disease at a Negative Predictive Value (NPV) of at least about 85%. In some embodiments, the biological sample is classified as having the disease at a Positive Predictive Value (PPV) of at least about 55%. In some embodiments, the method further comprises outputting a report on a computer screen that identifies the biological sample as negative or positive for the disease.

In some embodiments, the method further comprises filtering the biological sample by selecting for one or more of the following characteristics: a tissue type, a cytology type, a histology type, a collection method, a nucleic acid preservation method, a nucleic acid purification method, a library preparation method, a reagent utilized during processing, a sequencer apparatus employed, a sequencing software employed, or any combination thereof. In some embodiments, the filtering is performed before the assaying or before the comparing. In some embodiments, the filtering comprises employing a t-test, an analysis of variance (ANOVA) analysis, a Bayesian framework, a Gamma distribution, a Wilcoxon rank sum test, between-within class sum of squares test, a rank products method, a random permutation method, a threshold number of misclassification (TNoM), a bivariate method, a correlation based feature selection (CFS) method, a minimum redundancy maximum relevance (MRMR) method, a Markov blanket filter method, an uncorrelated shrunken centroid method, or any combination thereof.

In some embodiments, the tissue type comprises follicular carcinoma (FC), lymphocytic thyroiditis, follicular variant papillary thyroid carcinoma (FVPTC), papillary thyroid carcinoma (PTC), nodular hyperplasia (NHP), medullary thyroid carcinoma (MTC), Hurthle cell carcinoma (HCC), Hurthle cell adenoma (HCA), anaplastic thyroid carcinoma (ATC), follicular adenoma (FA), lymphocyte thyroiditis (LCT), benign follicular nodule (BFN), papillary thyroid carcinoma-tall cell variant (PTC-TCV), metastatic melanoma, metastatic renal carcinoma, metastatic breast carcinoma, parathyroid, metastatic B cell lymphoma, or any combination thereof. In some embodiments, the cytology type comprises benign, atypia/follicular lesion of undetermined significance (AUS/FLUS), follicular neoplasm/suspicion for a follicular neoplasm (FN/SFN), suspicious for malignancy (SFM), or malignant. In some embodiments, the histology type comprises benign or malignant.

In some embodiments, the collection method comprises a fine needle aspiration, a core needle biopsy, a tissue biopsy, a surgical resection, a collection method with anesthesia, a collection method without anesthesia, or any combination thereof. In some embodiments, the nucleic acid preservation method comprises use of RNeasy®, RNAProtect®, a TRIzol® product, RNALater®, QuickExtract™ RNA Extraction Kit, MasterPure™ RNA Purification Kit, or any combination thereof. In some embodiments, the nucleic acid purification method comprises use of a positive selection separation column, a negative selection separation column, a bead having a surface binding moiety, a molecular size separation column, a molecular charge separation column, or any combination thereof. In some embodiments, the library preparation method comprises use of Ovation® RNA Seq System, TruSeq® RNA Access Library Prep, or a combination thereof. In some embodiments, the sequencer apparatus comprises SOLiD®/Ion Torrent™ PGM™, Genome Analyzer, Hi Seq 2000, MiSeq, GS FLX Titanium, GS Junior, 454 sequencer, or combinations thereof.

In some embodiments, the biological sample is obtained from a subject. In some embodiments, the method further comprises prior to (a), obtaining the biological sample from the subject by fine needle aspiration. In some embodiments, the biological sample is cytologically ambiguous or suspicious. In some embodiments, the biological sample is about 20 micrograms or less. In some embodiments, the biological sample has an RNA Integrity Number (RIN) value of about 8.0 or less. In some embodiments, the biological sample comprises a fine needle aspirate sample (FNA), a core biopsy, a tissue biopsy, a surgical resection, or any combination thereof. In some embodiments, the biological sample comprises the FNA sample.

In some embodiments, the control sample comprises an FNA sample, a core biopsy, a tissue biopsy, a surgical resection, or any combination thereof. In some embodiments, the control sample comprises the FNA sample. In some embodiments, the control sample is obtained from a subject suspected of having or having been diagnosed with a disease. In some embodiments, the disease is thyroid cancer. In some embodiments, the one or more nucleic acid sequences of the control sample are associated with the noise-call. In some embodiments, the control sample is obtained from one or more of the following: a same subject as the biological sample, an independent biological sample, a tissue bank, a cell bank, a Clinical Laboratory Improvement Amendments (CLIA) lab, and a cell line.

In some embodiments, the biological sample comprises thyroid tissue, lung tissue, cardiac tissue, breast tissue, skin tissue, bone tissue, connective tissue, liver tissue, kidney tissue, pancreatic tissue, brain tissue, intestinal tissue, stomach tissue, esophagus tissue, oral tissue, facial tissue, dental tissue, spinal tissue, cervical tissue, uterine tissue, prostate gland tissue, or any combination thereof. In some embodiments, the method further comprises modifying the biological data set by removing the nucleic acid sequence from the biological dataset.

Another aspect of the present disclosure provides a computer system for processing a biological sample. The computer system may comprise a computer memory that stores a biological dataset comprising gene expression levels, sequence variant information, or a combination thereof corresponding to one or more nucleic acid sequences, which biological data set may be obtained by assaying the one or more nucleic acid sequences from the biological sample to obtain; and one or more computer processors operatively coupled to the computer memory and programmed to (i) compare the biological dataset to a second dataset comprising gene expression levels, sequence variant information, or a combination thereof corresponding to one or more nucleic acid sequences of a control sample; (ii) assign a call to the one or more nucleic acid sequences of the biological dataset based on the comparing of (i), wherein the call may be a no-call, a reference-call, or a noise-call; (iii) assign the noise-call to a nucleic acid sequence of the biological dataset; and (iv) upon assigning the noise-call to the nucleic acid sequence, (1) flag the nucleic acid sequence within the biological dataset, or (2) remove the nucleic acid sequence from the biological dataset, to produce the modified biological dataset.

In some embodiments, the biological dataset comprises the gene expression levels. In some embodiments, the biological dataset comprises the sequence variant information. In some embodiments, the second dataset comprises the gene expression levels. In some embodiments, the second dataset comprises the sequence variant information.

In some embodiments, the assigning further comprises applying (i) a DESeq Wald-test, a Limma test, a Fisher's extract test, or any combination thereof, (ii) a Hierarchical Ordered Partitioning And Collapsing Hybrid (HOPACH) cluster, or (iii) a combination thereof to the biological dataset. In some embodiments, the assigning further comprises applying the HOPACH cluster. In some embodiments, the assigning further comprises applying the Limma test and the DESeq Wald-test.

In some embodiments, the flag further comprises assigning a weighted value the nucleic acid sequence that is different from a value assigned to nucleic acid sequences of the biological dataset that are not assigned the noise-call. In some embodiments, the assaying comprises assaying a first portion of the biological sample separately from assaying a second portion of the biological sample. In some embodiments, the first portion is assayed at a different time, by a different operator, employing a different equipment type, employing a different reagent, or any combination thereof compared to the second portion. In some embodiments, the biological sample is obtained from a first source and a second source, and wherein the first source and the second source are different.

In some embodiments, the one or more computer processors determine a difference in expression level between the gene expression levels of the one or more nucleic acid sequences of the biological sample compared to the gene expression levels of one or more nucleic acid sequences of the control sample having at least about 90% homology to the one or more nucleic acid sequences from the biological sample. In some embodiments, when the difference in expression level for a given nucleic acid sequence of the biological sample is greater than about 10%, then the one or more computer processors assigns the noise-call to the given nucleic acid sequence in (c).

In some embodiments, the one or more computer processors determine a presence or an absence of a fusion in the one or more nucleic acid sequences of the biological sample compared to a presence or an absence of the fusion in one or more nucleic acid sequences of the control sample having at least about 90% homology to the one or more nucleic acid sequences of the biological sample. In some embodiments, when the fusion is present in a given nucleic acid sequence of the biological sample and is not present in a nucleic acid sequence of the control sample, then the one or more computer processors assigns the noise-call to the given nucleic acid sequence in (c). In some embodiments, the one or more computer processors employ a fusion panel that comprises less than about 200 fusions.

In some embodiments, the one or more computer processors determine a presence or an absence of a sequence variant, a sequence variant count number, or a combination thereof in the one or more nucleic acid sequences of the biological sample compared to a present or an absence of the sequence variant, the sequence variant count number, or a combination thereof in one or more nucleic acid sequences of the control sample having at least about 90% homology to the one or more nucleic acid sequences of the biological sample. In some embodiments, when the sequence variant is present in a given nucleic acid sequence of the biological sample and is not present in a nucleic acid sequence of the control sample, then the one or more computer processors assign the noise-call to the given nucleic acid sequence in (c). In some embodiments, when a raw count, a normalized count, or a number of counts of the sequence variant of a given nucleic acid sequence of the biological sample differs from a raw count, a normalized count or a number of counts of a sequence variant of a nucleic acid sequence in the control sample having at least about 90% homology to the given nucleic acid sequence of the biological sample, then the one or more computer processors assign the noise-call to the given nucleic acid sequence in (c). In some embodiments, the one or more computer processors employ a sequence variant panel that comprises less than about 900 sequence variants. In some embodiments, the biological sample is independent from the control sample.

In some embodiments, the gene expression levels of the biological dataset are measured by microarray, SAGE, blotting, RT-PCR, sequencing, and/or quantitative PCR. In some embodiments, the assaying comprises next generation sequencing of RNA (RNA Seq). In some embodiments, the sequence variants comprise a polymorphism, a mutation, a fusion, a splice variant, a copy number alteration, or any combination thereof. In some embodiments, the nucleic sequence assigned the noise-call in (iv) comprises a transcript degradation, an impartial fragmentation, an incomplete library preparation, a 3′ to 5′ bias, a polymerase processivity, a polymerase sequence bias, or any combination thereof. In some embodiments, the modified biological dataset comprises one or more nucleic acid sequences assigned a no-call, a reference call, or a combination thereof. In some embodiments, at least about 70% of the one or more nucleic acid sequences of the biological sample have at least about 90% sequence homology to a nucleic acid sequence of the one or more nucleic acid sequences of the control sample. In some embodiments, the control sample comprises a housekeeping gene.

In some embodiments, the one or more nucleic acid sequences of the biological dataset are less than about 10 nucleic acid sequences. In some embodiments, the one or more nucleic acid sequences of the control sample comprise less than about 900 nucleic acid sequences. In some embodiments, the nucleic acid sequence assigned the noise-call in (iv) comprises at least about 90% sequence homology to BRAF, HRAS, KRAS, NRAS, TSFIR, or RET, or any fragment thereof, or any combination thereof. In some embodiments, the nucleic acid sequence assigned the noise-call in (iv) comprises at least about 90% sequence homology to TSHR, RET, NRAS, TP53, PAX8, FAT1, VT11A, BRAF, IIRAS, or KKAS, or any fragment thereof, or any combination thereof.

In some embodiments, the one or more computer processors employ the modified biological dataset to train a trained algorithm. In some embodiments, the trained algorithm classifies the biological sample as negative for a disease. In some embodiments, the trained algorithm employs a Support Vector Machine (SVM) model, a Random Forest (RF) model, a Least Absolute Shrinkage and Selection Operator (LASSO) model, an Ensemble 1 model, a Penalized Logistic Regression (PLR) model, a Classification And Regression Trees (CART) model, or any combination thereof. In some embodiments, the disease is cancer. In some embodiments, the cancer is thyroid cancer.

In some embodiments, the biological sample is classified as negative for the disease at an accuracy of at least about 80%. In some embodiments, the biological sample is classified as negative for the disease at a specificity of at least about 70%. In some embodiments, the biological sample is classified as negative for the disease at a sensitivity of at least about 70%. In some embodiments, the biological sample is classified having the disease with a Negative Predictive Value (NPV) of at least about 85%. In some embodiments, the biological sample is classified as having the disease with a Positive Predictive Value (PPV) of at least about 55%.

In some embodiments, the computer system further comprises a computer screen, and wherein the computer system outputs a report on the computer screen that identifies the biological sample as negative for the disease. In some embodiments, the one or more computer processors filter the biological sample by selecting for one or more of the following characteristics: a tissue type, a cytology type, a histology type, a collection method, a nucleic acid preservation method, a nucleic acid purification method, a library preparation method, a reagent utilized during processing, a sequencer apparatus employed, a sequencing software employed, or any combination thereof.

In some embodiments, the one or more computer processors filter the biological sample before the assaying. In some embodiments, the one or more computer processors employ a t-test, an analysis of variance (ANOVA) analysis, a Bayesian framework, a Gamma distribution, a Wilcoxon rank sum test, between-within class sum of squares test, a rank products method, a random permutation method, a threshold number of misclassification (TNoM), a bivariate method, a correlation based feature selection (CFS) method, a minimum redundancy maximum relevance (MRMR) method, a Markov blanket filter method, an uncorrelated shrunken centroid method, or any combination thereof to filter the biological sample.

In some embodiments, the tissue type comprises follicular carcinoma (FC), lymphocytic thyroiditis, follicular variant papillary thyroid carcinoma (FVPTC), papillary thyroid carcinoma (PTC), nodular hyperplasia (NHP), medullary thyroid carcinoma (MTC), Hurthle cell carcinoma (HCC), Hurthle cell adenoma (HCA), anaplastic thyroid carcinoma (ATC), follicular adenoma (FA), lymphocyte thyroiditis (LCT), benign follicular nodule (BFN), papillary thyroid carcinoma-tall cell variant (PTC-TCV), metastatic melanoma, metastatic renal carcinoma, metastatic breast carcinoma, parathyroid, metastatic B cell lymphoma, or any combination thereof. In some embodiments, the cytology type comprises benign, atypia/follicular lesion of undetermined significance (AUS/FLUS), follicular neoplasm/suspicion for a follicular neoplasm (FN/SFN), suspicious for malignancy (SFM), or malignant. In some embodiments, the histology type comprises benign or malignant.

In some embodiments, the collection method comprises a fine needle aspiration, a core needle biopsy, a tissue biopsy, a surgical resection, a collection method with anesthesia, a collection method without anesthesia, or any combination thereof. In some embodiments, the biological sample is obtained from a subject. In some embodiments, the biological sample is cytologically ambiguous or suspicious. In some embodiments, the biological sample is about 20 micrograms or less. In some embodiments, the biological sample has an RNA Integrity Number (RIN) value of about 8.0 or less. In some embodiments, the biological sample comprises a fine needle aspirate sample (FNA), a core biopsy, a tissue biopsy, a surgical resection, or any combination thereof. In some embodiments, the biological sample comprises the FNA sample.

In some embodiments, the control sample comprises an FNA sample, a core biopsy, a tissue biopsy, a surgical resection, or any combination thereof. In some embodiments, the control sample comprises the FNA sample. In some embodiments, the biological sample comprises thyroid tissue, lung tissue, cardiac tissue, breast tissue, skin tissue, bone tissue, connective tissue, liver tissue, kidney tissue, pancreatic tissue, brain tissue, intestinal tissue, stomach tissue, esophagus tissue, oral tissue, facial tissue, dental tissue, spinal tissue, cervical tissue, uterine tissue, prostate gland tissue, or any combination thereof.

In some embodiments, the control sample is obtained from a subject suspected of having or having been diagnosed with a disease. In some embodiments, the disease is thyroid cancer. In some embodiments, the one or more nucleic acid sequences of the control sample are associated with the noise-call. In some embodiments, the control sample is obtained from one or more of the following: a same subject as the biological sample, an independent biological sample, a tissue bank, a cell bank, a Clinical Laboratory Improvement Amendments (CLIA) lab, and a cell line.

Another aspect of the present disclosure provides a non-transitory computer-readable medium. The non-transitory computer-readable medium may comprise machine-executable code that, upon execution by one or more computer processors, implements a method for processing a biological sample. In some embodiments, the method comprises: (a) assaying one or more nucleic acid sequences from the biological sample to obtain a biological dataset comprising gene expression levels, sequence variant information, or a combination thereof corresponding to the one or more nucleic acid sequences; (b) comparing the biological dataset assayed in (a) to a second dataset comprising gene expression levels, sequence variant information, or a combination thereof corresponding to one or more nucleic acid sequences of a control sample; (c) assigning a call to the one or more nucleic acid sequences of the biological dataset based on the comparing of (b), wherein the call is a no-call, a reference-call, or a noise-call; (d) assigning the noise-call to a nucleic acid sequence of the biological dataset; and (e) upon assigning the noise-call to the nucleic acid sequence, (i) flagging the nucleic acid sequence within the biological dataset, or (ii) removing the nucleic acid sequence from the biological dataset, to produce the modified biological dataset.

Another aspect of the present disclosure provides a method of diagnosing a genetic disorder or cancer. The method may comprise (a) obtaining a biological sample comprising gene expression products; (b) detecting the gene expression products of the biological sample; (c) comparing to an amount in a control sample, an amount of one or more gene expression products in the biological sample to determine the differential gene expression product level between the biological sample and the control sample; (d) classifying the biological sample by inputting the one or more differential gene expression product levels to a trained algorithm; and (e) identifying the biological sample as positive for a genetic disorder or cancer if the trained algorithm classifies the sample as positive for the genetic disorder or cancer at a specified confidence level. In some embodiments, technical factor variables are removed from data based on differential gene expression product level and normalized prior to and during classification.

In some embodiments, the gene expression product is mRNA. In some embodiments, the RNA has an RNA integrity number (RIN) of 2.0 or more. In some embodiments, the RNA with an RNA integrity number (RIN) of equal to or less than 5.0 is used for multi-gene microarray analysis. In some embodiments, multiple datasets based on differential gene expression product levels are joined. In some embodiments, a statistical method are used for training and testing a classifier, the statistical method are selected from the group consisting of support vector machines (SVM), linear discriminant analysis (LDA), K-nearest neighbor analysis (KNN), and random forest (RF). In some embodiments, the sample is obtained via one or more of the following: needle aspiration, fine needle aspiration, core needle biopsy, vacuum assisted biopsy, large core biopsy, incisional biopsy, excisional biopsy, punch biopsy, shave biopsy, or skin biopsy. In some embodiments, the sample is a pre-operative specimen. In some embodiments, the sample is a post-operative specimen. In some embodiments, the sample comprises thyroid tissue. In some embodiments, detecting the gene expression products of the biological test sample is performed by measuring mRNA. In some embodiments, mRNA is measured by one or more of the following: microarray, SAGE, blotting, RT-PCR, or quantitative PCR. In some embodiments, the normal sample is obtained from one or more of the following: the same individual as the test sample, a different individual from the test sample, a tissue or cell bank. In some embodiments, the normal control sample gene expression product amounts are from a database. In some embodiments, the method distinguishes thyroid carcinoma from benign thyroid diseases. In some embodiments, the method further comprises providing a suggested therapeutic intervention. In some embodiments, the results of the expression analysis provide a statistical confidence level above 90% that a given diagnosis is correct. In some embodiments, the method further comprises the step of performing a cytological analysis on a portion of the biological sample following step (a) to obtain a preliminary diagnosis. In some embodiments, the diagnosis of the genetic disorder or cancer has a specificity of at least 70%. In some embodiments, the diagnosis of the genetic disorder or cancer has a sensitivity of at least 70%. In some embodiments, the diagnosis of the genetic disorder or cancer has an accuracy of at least 90%. In some embodiments, multiple datasets on the biological sample is joined.

Another aspect of the present disclosure provides an algorithm for diagnosing a genetic disorder or cancer. The algorithm may comprise (a) determining the level of gene expression products in a biological sample; (b) deriving the composition of cells in the biological sample based on the expression levels of cell-type specific markers in the sample; (c) removing technical variables prior to and during classification of the biological sample; (d) correcting or normalizing the gene product levels determined in step (a) based on the composition of cells determined in step (b); and (e) classifying the biological sample as positive for a genetic disorder or cancer.

In some embodiments, the cell-types comprise one or more of the following: red blood cell, platelet, medullary cell, follicular cell, smooth muscle cell, macrophage, and lymphocyte. In some embodiments, the sample comprises thyroid tissue. In some embodiments, the level of gene expression products is determined by measuring mRNA. In some embodiments, the mRNA has an RNA integrity number (RIN) of 2.0 or more. In some embodiments, mRNA is measured by one or more of the following: microarray, SAGE, blotting, RT-PCR, or quantitative PCR. In some embodiments, the algorithm distinguishes thyroid follicular carcinoma from thyroid follicular adenoma. In some embodiments, the algorithm further comprises identifying the test sample as cancerous or positive for a genetic disorder if a trained algorithm classifies the sample as cancerous or positive for a genetic disorder at a specified confidence level. In some embodiments, the results of the expression product analysis provide a statistical probability above 90% that a given diagnosis is correct. In some embodiments, the diagnosis of the genetic disorder or cancer has a specificity of at least 70%. In some embodiments, the diagnosis of the genetic disorder or cancer has a sensitivity of at least 70%. In some embodiments, multiple datasets on the biological sample are joined.

Another aspect of the present disclosure provides a method of diagnosing thyroid cancer. The method may comprise (a) obtaining a biological sample comprising gene expression products, wherein the biological sample may comprise a fine needle aspirate (FNA) of thyroid tissue from a subject; (b) assaying by sequencing, array hybridization, or nucleic acid amplification the gene expression products of the biological sample, which gene expression products may be associated with a benign or malignant thyroid condition; (c) comparing to an amount in a control sample, an amount of one or more gene expression products in the biological sample to determine one or more differential gene expression product levels between the biological sample and the control sample; (d) classifying the biological sample by inputting the one or more differential gene expression product levels to a trained algorithm; and (e) outputting a report on a computer screen that may identify the biological sample as negative for the thyroid cancer if the trained algorithm classifies the biological sample as negative for the thyroid cancer at a specified confidence level. In some embodiments, the trained algorithm classifies biological samples as negative for thyroid cancer at an accuracy of at least 90%. In some embodiments, a plurality of technical factor variables are removed from data based on one or more of the differential gene expression product levels and normalized prior to or during classification. In some embodiments, the plurality of technical factor variables are selected from the group consisting of a collection source, a collection method, a collection media, a RNA integrity number, a whole transcriptome amplification yield, a sense strand yield, a hybridization site, a hybridization quality and an experiment batch.

In some embodiments, the gene expression products are mRNA. In some embodiments, the mRNA has an RNA integrity number (RIN) of 2.0 or more. In some embodiments, a sample of the mRNA is used for multi-gene microarray analysis. In some embodiments, the biological sample has an RNA integrity number (RIN) of equal to or less than 5.0. In some embodiments, the trained algorithm is trained with multiple datasets of differential gene expression product levels obtained from training samples. In some embodiments, the classifying the biological sample is done by a classifier and a statistical method is used for training and testing the classifier. In some embodiments, the statistical method is selected from the group consisting of support vector machines (SVM), linear discriminant analysis (LDA), K-nearest neighbor analysis (KNN), and random forest (RF). In some embodiments, the assaying the gene expression products is performed by measuring mRNA. In some embodiments, the mRNA is measured by one or more of the following: microarray, SAGE, blotting, RT-PCR, or quantitative PCR. In some embodiments, the control sample is obtained from one or more of the following: the same subject as the biological sample, a different subject from the biological sample, and a tissue or cell bank. In some embodiments, the thyroid tissue has a benign thyroid disease and the trained algorithm does not classify the biological sample comprising the FNA of thyroid tissue as positive for cancer.

Another aspect of the present disclosure provides a method of diagnosing thyroid cancer. The method may comprise (a) obtaining a biological sample comprising gene expression products, wherein the biological sample may comprise a fine needle aspirate (FNA) sample from a subject; (b) assaying by sequencing, array hybridization, or nucleic acid amplification the gene expression products of the biological sample, which gene expression products may be associated with a benign or malignant thyroid condition; (c) comparing to an amount in a control sample, an amount of one or more gene expression products in the biological sample to determine one or more differential gene expression product levels between the biological sample and the control sample; (d) classifying the biological sample by inputting the one or more differential gene expression product levels to a trained algorithm; (e) identifying the biological sample as negative for the thyroid cancer if the trained algorithm classifies the biological sample as negative for the thyroid cancer at a specified confidence level; and (f) providing a report on a computer screen with a suggested therapeutic intervention. In some embodiments, the trained algorithm may classify biological samples as negative for thyroid cancer at an accuracy of at least 90%. In some embodiments, a plurality of technical factor variables are removed from data based on one or more of the differential gene expression product levels and are normalized prior to or during classification. In some embodiments, the plurality of technical factor variables are selected from the group consisting of a collection source, a collection method, a collection media, a RNA integrity number, a whole transcriptome amplification yield, a sense strand yield, a hybridization site, a hybridization quality, and an experiment batch. In some embodiments, the specified confidence level are above 90% for at least two subtypes of thyroid cancer.

Another aspect of the present disclosure provides a method of diagnosing thyroid cancer. The method may comprise (a) obtaining a biological sample comprising gene expression products, wherein cytological analysis may have been performed on a portion of the biological sample to obtain a preliminary diagnosis indicating that the cytological analysis is ambiguous, and wherein the biological sample may comprise a fine needle aspirate (FNA) sample from a subject; (b) assaying by sequencing, array hybridization, or nucleic acid amplification the gene expression products of the biological sample, which gene expression products may be associated with a benign or malignant thyroid condition; (c) comparing to an amount in a control sample, an amount of one or more gene expression products in the biological sample to determine one or more differential gene expression product levels between the biological sample and the control sample; (d) classifying the biological sample by inputting the one or more differential gene expression product levels to a trained algorithm; and (e) outputting a report on a computer screen that may identify the biological sample as negative for the thyroid cancer if the trained algorithm classifies the biological sample as negative for the thyroid cancer at a specified confidence level. In some embodiments, the trained algorithm classifies biological samples as negative for thyroid cancer at an accuracy of at least 90%. In some embodiments, technical factor variables are removed from data based on one or more of the differential gene expression product levels and normalized prior to or during classification. In some embodiments, the biological sample is classified at a specificity of at least 70%. In some embodiments, the biological sample is classified at a sensitivity of at least 70%.

Another aspect of the present disclosure provides a method for classifying a thyroid cancer. The method may comprise (a) assaying by sequencing, array hybridization, or nucleic acid amplification to determine a level of gene expression products in a biological sample, wherein the biological sample may comprise a fine needle aspirate (FNA) sample from a subject, and wherein the gene expression products may be associated with a benign or malignant thyroid condition; (b) deriving a composition of cells in the biological sample based on expression levels of cell-type specific markers in the biological sample; (c) removing a plurality of technical factor variables prior to or during classification of the biological sample; (d) correcting or normalizing gene product levels determined in step (a) based on the composition of cells determined in step (b); (e) classifying the biological sample as positive or negative for the thyroid cancer using a trained algorithm that may classify biological samples as negative for the thyroid cancer at an accuracy of at least 90%; and (f) outputting a report on a computer screen that may be indicative of a classification of the biological sample as positive or negative for the thyroid cancer.

In some embodiments, the biological sample is classified at a specificity that is greater than 80%. In some embodiments, the biological sample is classified at a sensitivity that is greater than 60%. In some embodiments, the biological sample is classified at a specificity that is greater than 90%. In some embodiments, the biological sample is classified at a sensitivity that is greater than 80%. In some embodiments, the biological sample comprises thyroid tissue. In some embodiments, the plurality of technical factor variables comprise two or more technical factor variables selected from the group consisting of a collection source, a collection method, a collection media, a whole transcriptome amplification yield, a sense strand yield, a hybridization site, a hybridization quality and an experiment batch. In some embodiments, the plurality of technical factor variables comprise three or more technical factor variables selected from the group consisting of a collection source, a collection method, a collection media, a whole transcriptome amplification yield, a sense strand yield, a hybridization site, a hybridization quality and an experiment batch. In some embodiments, the plurality of technical factor variables comprise four or more technical factor variables selected from the group consisting of a collection source, a collection method, a collection media, a whole transcriptome amplification yield, a sense strand yield, a hybridization site, a hybridization quality and an experiment batch. In some embodiments, the plurality of technical factor variables are selected from the group consisting of a collection media, a hybridization site, a hybridization quality, a whole transcriptome amplification yield, a sense strand yield and an experiment batch. In some embodiments, the plurality of technical factor variables are selected from the group consisting of a collection media, a hybridization site, a hybridization quality, a whole transcriptome amplification yield, and a sense strand yield. In some embodiments, the plurality of technical factor variables is removed from the data by adjusting the data for variation due to the plurality of technical factor variables. In some embodiments, the biological sample is cytologically ambiguous or suspicious.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows variant information (categorical) and gene expression levels (quantitative) of nucleic acid sequences at specific genomic locations in a fine needle aspirate (FNA) sample list.

FIG. 2 shows a tunneling bloc biopsy (TBB) sample list.

FIG. 3 shows sample composition and reagent lot of each experimental run.

FIG. 4 shows sequencing error examples compared to a reference sequence.

FIG. 5 shows a computer control system that is programmed or otherwise configured to implement methods provided herein.

FIG. 6 shows sample cohort characteristics of a feasibility study in Example 2.

FIG. 7 shows a performance summary of a feasibility study in Example 2.

FIG. 8 shows a classifier development for a feasibility study in Example 2.

FIG. 9 shows Hierarchical Ordered Partitioning And Collapsing Hybrid (HOPACH) clustering in a training set of a feasibility study in Example 2 showing clustering on a Top 2000 expression genes.

FIG. 10 shows HOPACH clustering in a training set of a feasibility study in Example 2 showing clustering on a Top 1402 variants.

FIG. 11 shows individual scores of validation samples in the feasibility study in Example 2.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. Humans can be more than about 1, 2, 5, 10, 20, 30, 40, 50, 60, 65, 70, 75, or about 80 years of age. The subject may have or be suspected of having a disease, such as cancer. The subject may be a patient, such as a patient being treated for a disease, such as a cancer patient. The subject may be predisposed to a risk of developing a disease such as cancer. The subject may be in remission from a disease, such as a cancer patient. The subject may be healthy.

The term “disease,” as used herein, generally refers to any abnormal or pathologic condition that affects a subject. Examples of a disease include cancer, such as, for example, thyroid cancer, parathyroid cancer, lung cancer, skin cancer, breast cancer, colon cancer, pancreatic cancer and others. The disease may be treatable or non-treatable. The disease may be terminal or non-terminal. The disease can be a result of inherited genes, environmental exposures, or any combination thereof. The disease can be cancer, a genetic disease, a proliferative disorder, or others as described herein. The disease may be cancer such as thyroid cancer. A thyroid cancer may be a subtype such as follicular adenoma (FA), nodular hyperplasia (NHP), lymphocytic thyroiditis (LCT), Hurthie cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), anaplastic thyroid carcinoma (ATC), renal carcinoma (RCC), breast carcinoma (BCA), melanoma (MMN), B cell lymphoma (BCL), parathyroid (PTA), or hyperplasia papillary carcinoma (HPC).

The term “sequence variant information,” “sequence variation,” “sequence alteration” or “allelic variant,” as used herein, generally refer to a specific change or variation in relation to a reference sequence, such as a genomic deoxyribonucleic acid (DNA) reference sequence, a coding DNA reference sequence, or a protein reference sequence, or others. The reference DNA sequence can be obtained from a reference database. A sequence variant may affect function. A sequence variant may not affect function. A sequence variant can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a sequence variation can include a substitution, a deletion, an insertion, an inversion, or a conversion in one or more nucleotides or one or more amino acids. A sequence variant may be a point mutation. A sequence variant may be a fusion gene. A fusion pair or a fusion gene may result from a sequence variant, such as a translocation, an interstitial deletion, a chromosomal inversion, or any combination thereof. A sequence variation can constitute variability in the number of repeated sequences, such as triplications, quadruplications, or others. For example, a sequence variation can be an increase or a decrease in a copy number associated with a given sequence (i.e., copy number variation, or CNV). A sequence variation can include two or more sequence changes in different alleles or two or more sequence changes in one allele. A sequence variation can include two different nucleotides at one position in one allele, such as a mosaic. A sequence variation can include two different nucleotides at one position in one allele, such as a chimeric. A sequence variant may be present in a malignant tissue. A sequence variant may be present in a benign tissue. Absence of a variant may indicate that a tissue or sample is benign. As an alternative, absence of a variant may not indicate that a tissue or sample is benign.

The term “mutation panel,” as used herein, generally refers to a panel designating a specified number of genomic sites and fusion pairs that are to be detected (or interrogated). For example, a mutation panel may comprise a fusion panel. A mutation panel may comprise a sequence variant panel. A mutation panel may comprise less than 10 genomic sites, less than 10 sequence variants, less than 10 fusion pairs, or any combination thereof. A mutation panel may comprise one or more genomic sites, one or more sequence variants, and one or more fusion pairs. A mutation panel may comprise more than about 1, 2, 3, 4, 5, 10, 20, 30, 40, 50, 100, 200, 300, 400, or 500 genomic sites. A mutation panel may comprise more than about 15 genomic sites. A mutation panel may comprise more than about 100 genomic sites. A mutation panel may comprise more than about 200 genomic sites. A mutation panel may comprise more than about 500 genomic sites. A mutation panel may comprise more than about 1000 genomic sites. A mutation panel may comprise more than about 2000 genomic sites. A mutation panel may comprise more than about 3000 genomic sites. A mutation panel may comprise more than about 1 or 2 fusion pairs. A mutation panel may comprise more than about 5 fusion pairs. A mutation panel may comprise more than about 10 fusion pairs. A mutation panel may comprise more than about 15 fusion pairs. A mutation panel may comprise more than about 20 fusion pairs. A mutation panel may comprise more than about 25 fusion pairs. A mutation panel may comprise more than about 1 or 2 sequence variants. A mutation panel may comprise more than about 5 sequence variants. A mutation panel may comprise more than about 10 sequence variants. A mutation panel may comprise more than about 15 sequence variants. A mutation panel may comprise more than about 20 sequence variants. A mutation panel may comprise more than about 25 sequence variants. A mutation panel, such as a sequence variant panel, may comprise less than about 900, 800, 700, 600, 500, 400, 300, 200, 100, 50, 20, 10 sequence variants or less.

The term “modified biological dataset,” as used herein, generally refers to a biological dataset that has been modified. The biological dataset may comprise gene expression levels, sequence variant information, or a combination thereof. The modified biological dataset may be modified by flagging a particular gene corresponding to a gene expression level or presence or absence of a sequence variant. The modified biological dataset may be modified by removing a particular gene corresponding to a gene expression level or presence or absence of a sequence variant. The modified biological dataset may be modified by weighting a particular gene differently from other genes in the dataset.

The term “no-call,” as used herein, generally refers to a label or an identifier assigned to a particular gene, set of genes, sequence of plurality of sequences in a modified dataset based on a comparison to a control sample of gene expression levels, sequence information (e.g., sequence variant information), or a combination thereof. The gene expression levels may include transcript levels. The sequence information may include DNA sequence information. A no-call may indicate that there may be insufficient data or insufficient certainty to label or identify the particular gene as a reference-call or a noise-call. A no-call may be a lack of information about the particular gene in the modified dataset.

The term “reference-call,” as used herein, generally refers to a label or an identifier assigned to a particular gene, set of genes, sequence or a plurality of sequences in a modified dataset based on a comparison to a control sample of gene expression levels, sequence information (e.g., sequence variant information), or a combination thereof. A reference-call may indicate a biological truth or diagnostic truth, the opposite of a noise-call showing a false biological or diagnostic effect because of a poor signal to noise ratio. A reference-call may be indicative of a true biological effect (e.g., a disease, such as thyroid or lung cancer). A reference-call may be indicative of a given gene, set of genes, sequence or plurality of sequences that are directly associated with a disease, such as, for example cancer (e.g., thyroid cancer). In some examples, a reference-call is indicative of a given gene, set of genes, sequence or plurality of sequences that are directly associated with a corresponding reference gene, set of genes, sequence of plurality of sequences, such as, for example, what may be identified as normal or a healthy control.

The term “noise-call,” as used herein, generally refers to a label or an identifier assigned to a particular gene, set of genes, sequence of plurality of sequences in a modified dataset based on a comparison to a control sample of gene expression levels, sequence information (e.g., sequence variant information), or a combination thereof. A noise-call may indicate that a noise level of a particular gene, set of genes, sequence or a plurality of sequences to which it is assigned, may be too high and may mask a true biological effect or that that particular gene, set of genes, sequence or a plurality of sequences may be a less desirable gene, set of genes, sequence or a plurality of sequences for input into a trained algorithm or classifier. A noise-call may be assigned to a gene, set of genes, sequence or a plurality of sequences if an expression level of the gene, set of genes, sequence or a plurality of sequences is more than about 1%, 5%, 10%, 20%, 30%, 40%, or 50% different than an expression level of the same gene in a control sample, or if the expression level cannot be differentiated from a signal noise level (e.g., background noise). A noise-call may be assigned to a gene, set of genes, sequence or a plurality of sequences if a sequence or sequence variant of the gene, set of genes, sequence or a plurality of sequences is present in the modified dataset and is not presence in a control sample.

### Methods and Systems for Processing a Biological Sample

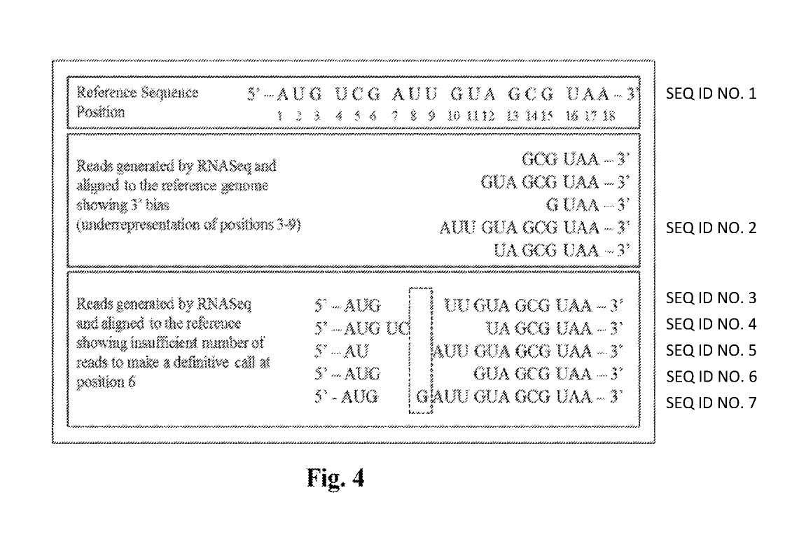

Categorical sequence determination may be independent of the methods used to resolve the sequence. In practice however both the categorical (e.g., variant) and quantitative (e.g., gene expression) determination of nucleotide sequences at specific genomic locations can be susceptible to measurement errors (FIG. 4) due to a variety of reasons including but not limited to; transcript degradation, impartial fragmentation, incomplete library preparation, 3′ to 5′ bias, polymerase processivity and/or polymerase sequence bias, and any other process that may randomly or may selectively preclude a genomic region from being purified, amplified, fragmented, barcoded, labeled, enriched, filtered, or detected relative to the true proportion of other transcripts present in the original sample at the instant the specimen was collected (including both artificial under and over representation of sequence).

Because RNASeq may rely in part on read depth to make a categorical determination of sequence at a given genomic position, this process relies on quantitation, and is thus prone to batch effects. Reagent lot-dependent variation may be an undesirable and costly consequence of running nucleic amplification and sequencing assays. Quantitatively and qualitatively the results obtained from a sample processed with different lots of the same reagents may vary drastically. This in turn may directly impact any downstream evaluation and performance of gene signatures or gene panels that may be identified or may be obtained through machine learning efforts. In order for classifier predictions to be accurate and reproducible, reduction or elimination of batch effects that are independent of biology is important.

The methods and systems disclosed herein process biological datasets, such that nucleic acid sequences of the biological datasets that may be highly susceptible to batch effects are selected against, flagged, removed from the dataset, “black listed” or weighed differently in subsequent machine learning classification efforts. Any sequences that may be highly susceptible to batch effects may be reduced or eliminated, thereby increasing the signal to noise used in subsequent analyses. “Blacklisting” sequences as susceptible to batch effects may modify or transform the originally obtained sequencing data in a manner that may render the resulting sequences (both “blacklisted” and not-blacklisted) as distinct sequence populations. After modification of a biological dataset, a minimum of two sequence populations may exist, but many more may be possible, as these sequence populations may be empirically derived and may be expected to be unique for any combination of 1) tissue type (such as thyroid tissue, brain tissue, lung tissue); 2) cytology type; 3) histology type; 4) collection method (such as FNA, surgical, with or without anesthesia, or others); 5) nucleic acid preservation method (such as RNAPortect, Trizol, or others); 6) nucleic acid purification method (such as columns, beads, or others); 7) library preparation method (such as Nugen's Ovation, Illumina's RNA Access, or others); 8) any and/or all reagents used during laboratory processing of the samples; 9) sequencer apparatus used (such as Illumina's Mi Seq, HiSeq, or others); and/or 10) sequencing software used for alignment, variant calling, or others. Once all the sequences that may be susceptible to batch effects are identified, these can be categorized into discreet groups, flagged individually, and/or weighed differently such that downstream analyses may take this information into account in order to interpret 1) gene expression 2) ascertain variant calling results or 3) in order to increase the signal-to-noise ratio used during training of a classifier, a trained algorithm, or machine learning algorithms. It may be expected that the methods and systems described herein may allow for novel and/or more accurate ways to train tissue classifiers and to predict diagnostic outcomes using gene expression classifiers, while simultaneously avoiding the labor and cost intensive process of “reagent lot calibration” that is the current standard practiced commercially today.

The present disclosure provides methods and systems for processing a biological sample, such as modifying a biological dataset obtained from the biological sample. The biological sample may be obtained from a subject. The biological sample may comprise a fine needle aspirate, a tissue biopsy, surgical resection, or combinations thereof. The biological sample may be obtained at one or more times, one or more locations, using one or more buffers or reagents, or any combination thereof. Such methods can comprise assaying one or more nucleic acid sequences from the biological sample. The assaying may obtain a biological dataset comprising gene expression levels, sequence variant information, or a combination thereof corresponding to the one or more nucleic acid sequences. The assaying may comprise RNA sequencing, RT-PCR, array hybridization, nucleic acid sequencing, nucleic acid amplification, Next lien sequencing, microarray analysis, or others.

Next, the biological dataset is compared to a second dataset. The second dataset may comprise a control sample. The second dataset may also comprise gene expression levels, sequence variant information, or a combination thereof corresponding to one or more nucleic acid sequences of a control sample. The control sample may comprise a fine needle aspirate, a tissue biopsy, a surgical resection, or combinations thereof. The control sample may comprise a gene having at least about 90% sequence homology to a gene of the biological dataset. The comparing may be implemented by a computer processor. One or more gene expression levels of the biological dataset may be compared to one or more gene expression levels of the control sample. One or more sequence variants of the biological dataset may be compared to one or more gene expression levels of the control sample.

Next, a call may be assigned to one or more nucleic acid sequences of the biological dataset. A call may be assigned to each nucleic acid sequence of the biological dataset. A call may be assigned to a subset of the nucleic acid sequences of the biological sample. The call that is assigned may be based on the comparison. For example, a call may be based upon a comparison between a gene expression level of the biological dataset and a gene expression level of the control sample. A call may be assigned by applying a DESeq Wald-test, a Limma test, a Fisher's extract test, a Hierarchical Ordered. Partitioning and Collapsing Hybrid (HOPACH) c er, or any combination thereof. A call may be assigned by applying a test or a cluster or a combination thereof.

The call may be a no-call, a reference-call, or a noise-call. A no-call may be assigned for insufficient or inconclusive result. A reference-call may be assigned for a gene having a gene expression level that is a biological or diagnostic truth. A noise-call may be assigned to a gene having a gene expression level, a fusion, or a sequence variant that deviates from a biological truth or a control sample gene expression level. A noise-call may be assigned to a gene having a gene expression level, a fusion, or a sequence variant that may not be reflective of a true biological event, but may be a poor signal to noise ratio. A noise-call may be assigned to a gene that when input into a tissue classifier, may not yield a sufficient accurate, sensitive, or specific result, such as a clinical or diagnostic result.

Next, a noise-call may be assigned to a nucleic acid sequence of the biological dataset. Upon assigning the noise-call to the nucleic acid sequence, the biological dataset is modified. The biological dataset may be modified by flagging the nucleic acid sequence assigned the noise-call. The nucleic acid sequence may be flagged by assigned a weighted value to the nucleic acid sequence that is different than a value assigned other nucleic acid sequences of the modified biological dataset. The biological dataset may be modified by removing the nucleic acid sequence assigned the noise-call.

Comparing a gene expression level in the biological sample to a control sample may include determining a difference in expression level between the two. The gene expression level of a nucleic acid sequence in the biological sample may have at least 90% homology to the nucleic acid sequence of the control sample to which it is compared. The nucleic acid sequence of the control sample may have at least 91% sequence homology to the nucleic acid sequence of the biological sample. It may have 92% sequence homology. It may have 93% sequence homology. It may have 94% sequence homology. It may have 95% sequence homology. It may have 96% sequence homology. It may have 97% sequence homology. It may have 98% sequence homology. It may have 99% sequence homology.

When a difference in an expression level of a nucleic acid sequence between a control sample and a biological sample is greater than about 1%, 2%, 3%, 4%, 5%, 6%, 7%, 8%, 9%, 10%, 11%, 12%, 13%, 14%, 15%, 16%, 17%, 18%, 19%, 20% or more, nucleic acid sequence of the biological sample that corresponds to the gene expression level may be assigned a noise-call. When a fusion or sequence variant is present in the control sample and is not present in the biological sample, a noise-call may be assigned to the biological sample. When a fusion or sequence variant is present in the biological sample and is not present in the control sample, a noise-call may be assigned to the biological sample. When a difference in a raw count, a normalized count, or a count number of a sequence variant of a nucleic acid sequence between a control sample and a biological sample is greater than about 1%, 2%, 3%, 4%, 5%, 6%, 7%, 8%, 9%, 10%, 11%, 12%, 13%, 14%, 15%, 16%, 17%, 18%, 19%, 20% or more, the nucleic acid sequence of the biological sample that corresponds to the raw count, normalized count, or counter number may be assigned a noise-call.

The methods and systems of the present disclosure may modify a biological dataset. A modification to a biological dataset may include assigning a noise-call to one or more nucleic acid sequences from the biological dataset. A modification to a biological dataset may include flagging one or more nucleic acid sequences within the dataset A modification may include removing one or more nucleic acid sequences from the dataset. A modification may include assigning a weight one or more nucleic acid sequences that may be different than a weight assigned to other nucleic acid sequences in the biological dataset. A modification to a biological dataset may be based on a gene expression level, a fusion, a sequence information, or any combination thereof of the one or more nucleic acid sequences in the biological dataset. A nucleic acid sequence may be assigned a noise-call if a gene expression level, gene expression pattern, fusion, sequence information, or any combination thereof is not a biological truth or a true biological effect. For example, a first gene may have a fluctuating gene expression level depending on a reagent used or sequencer equipment used or other and a second gene may maintain a biological true gene expression level regardless of the reagent or sequencer equipment used. The second gene may be more desirable to include in a clinical diagnostic method or a trained algorithm. The first gene may be less desirable to include in a trained algorithm or in a clinical diagnostic method. The first gene may be removed from the biological dataset thereby modifying the dataset. A degree or a threshold of fluctuation in a gene expression level compared to a true biological gene expression level may determine whether a gene may or may not be assigned a noise-call. For example, a gene expression level that may deviate more than about 1%, 3%, 5%, 8%, 10%, 15%, 20% or more from a biological true expression level may be assigned a noise-call. In another example, a first gene may or may not have a sequence variant at a location in a nucleic acid sequence depending on a reagent used or sequencer equipment used and a second gene may maintain either a presence or an absence of the sequence variant regardless of the reagent or sequencer equipment used. The first gene may be assigned a noise-call and may be less desirable than the second gene to be included in training an algorithm and or a clinical diagnostic method.

### Samples

A sample obtained from a subject can comprise tissue, cells, cell fragments, cell organelles, nucleic acids, genes, gene fragments, expression products, gene expression products, gene expression product fragments or any combination thereof. A sample can be heterogeneous or homogenous. A sample can comprise blood, urine, cerebrospinal fluid, seminal fluid, saliva, sputum, stool, lymph fluid, tissue, or any combination thereof. A sample can be a tissue-specific sample such as a sample obtained from a thyroid tissue, skin, heart, lung, kidney, breast, pancreas, liver, muscle, smooth muscle, bladder, gall bladder, colon, intestine, brain, esophagus, or prostate.

A sample of the present disclosure can be obtained by various methods, such as, for example, needle aspiration, core needle biopsy, vacuum assisted biopsy, incisional biopsy, excisional biopsy, core biopsy, punch biopsy, shave biopsy, skin biopsy, or any combination thereof. The needle aspiration may be, for example, fine needle aspiration (FNA). Such needle aspirate (e.g., FNA) sample may be cytologically ambiguous or suspicious (or indeterminate).

A sample may be a biological sample, such as a sample obtained from a subject. A biological sample may be obtained by fine needle aspirate (FNA), core biopsy, surgical resection, or others, or a combination thereof. A sample may be a control sample. A control sample may be compared against a biological sample. A sample may be a training sample. A training sample may be employed to train a trained algorithm. A control sample may be obtained by fine needle aspirate, core biopsy, surgical resection, or others, or a combination thereof. A training sample may be obtained by fine needle aspirate, core biopsy, surgical resection, or others, or a combination thereof. A biological sample may be independent from a control sample. A training sample may be independent from a biological sample.

FNA, also referred to as fine needle aspirate biopsy (FNAB), or needle aspirate biopsy (NAB), is a method of obtaining a small amount of tissue from a subject. FNA can be less invasive than a tissue biopsy, which may require surgery and hospitalization of the subject to obtain the tissue biopsy. The needle of a FNA method can be inserted into a tissue mass of a subject to obtain an amount of sample for further analysis. In some cases, two needles can be inserted into the tissue mass. The FNA sample obtained from the tissue mass may be acquired by one or more passages of the needle across the tissue mass. In some cases, the FNA sample can comprise less than about 6×10⁶, 5×10⁶, 4×10⁶, 3×10⁶, 2×10⁶, 1×10⁶ cells or less. The needle can be guided to the tissue mass by ultrasound or other imaging device. The needle can be hollow to permit recovery of the FNA sample through the needle by aspiration or vacuum or other suction techniques.

Samples obtained using methods disclosed herein, such as an FNA sample, may comprise a small sample volume. A sample volume may be less than about 100 microliters (uL), 75 uL, 50 uL, 25 uL, 20 uL, 15 uL, 10 uL, 5 uL, 1 uL, 0.5 uL, 0.1 uL, 0.01 uL or less. The sample volume may be less than about 1 uL. The sample volume may be less than about 5 uL. The sample volume may be less than about 10 uL. The sample volume may be less than about 20 uL. The sample volume may be less than about 15 uL. The sample volume may be between about 1 uL and about 10 uL. The sample volume may be between about 10 uL and about 25 uL.

Samples obtained using methods disclosed herein, such as an FNA sample, may comprise small sample weights. The sample weight, such as a tissue weight, may be less than about 100 milligrams (mg), 75 mg, 50 mg, 25 mg, 20 mg, 15 mg, 10 mg, 9 mg, 8 mg, 7 mg, 6 mg, 5 mg, 4 mg, 3 mg, 2 mg, 1 mg, 0.5 mg, 0.1 mg or less. The sample weight may be less than about 20 mg. The sample weight may be less than about 10 mg. The sample weight may be less than about 5 mg. The sample weight may be between about 5 mg and about 20 mg. The sample weight may be between about 1 mg and about 5 ng.

Samples obtained using methods disclosed herein, such as FNA, may comprise small numbers of cells. The number of cells of a single sample may be less than about 10×10⁶, 5.5×10⁶, 5×10⁶, 4.5×10⁶, 4×10⁶, 3.5×10⁶, 3×10⁶, 2.5×10⁶, 2×10⁶, 1.5×10⁶, 1×10⁶, 0.5×10⁶, 0.2×10⁶, 0.1×10⁶ cells or less. The number of cells of a single sample may be less than about 5×10⁶ cells. The number of cells of a single sample may be less than about 4×10⁶ cells. The number of cells of a single sample may be less than about 3×10⁶ cells. The number of cells of a single sample may be less than about 2×10⁶ cells. The number of cells of a single sample may be between about 1×10⁶ and about 5×10⁶ cells. The number of cells of a single sample may be between about 1×10⁶ and about 10×10⁶ cells.

Samples obtained using methods disclosed herein, such as FNA, may comprise small amounts of deoxyribonucleic acid (DNA) or ribonucleic acid (RNA). The amount of DNA or RNA in an individual sample may be less than about 500 nanograms (ng), 400 ng, 300 ng, 200 ng, 100 ng, 75 ng, 50 ng, 45 ng, 40 ng, 35 ng, 30 ng, 25 ng, 20 ng, 15 ng, 10 ng, 5 ng, 1 ng, 0.5 ng, 0.1 ng, or less. The amount of DNA or RNA may be less than about 200 ng. The amount of DNA or RNA may be less than about 100 ng. The amount of DNA or RNA may be less than about 40 ng. The amount of DNA or RNA may be less than about 25 ng. The amount of DNA or RNA may be less than about 15 ng. The amount of DNA or RNA may be between about 1 ng and about 25 ng. The amount of DNA or RNA may be between about 5 ng and about 50 ng.

RNA yield or RNA amount of a sample can be measured in nanogram to microgram amounts. An example of an apparatus that can be used to measure nucleic acid yield in the laboratory is a NANODROP® spectrophotometer, CUBIT® fluorometer, or QUANTUS™ fluorometer. The accuracy of a NANODROP® measurement may decrease significantly with very low RNA concentration. Quality of data obtained from the methods described herein can be dependent on INA quantity. Meaningful gene expression or sequence variant data or others can be generated from samples having a low or un-measurable RNA concentration as measured by NANODROP®. In some cases, gene expression or sequence variant data or others can be generated from a sample having an unmeasurable RNA concentration.

The methods as described herein can be performed using samples with low quantity or quality of polynucleotides, such as DNA or RNA. A sample with low quantity or quality of RNA can be for example a degraded or partially degraded tissue sample. A sample with low quantity or quality of RNA may be a fine needle aspirate (FNA) sample. The RNA quality of a sample can be measured by a calculated RNA Integrity Number (RIN) value. The RIN value is an algorithm for assigning integrity values to RNA measurements. The algorithm can assign a 1 to 10 RIN value, where an RIN value of 10 can be completely intact RNA. A sample as described herein that comprises RNA can have an RIN value of about 9.0, 8.0, 7.0, 6.0, 5.0, 4.0, 3.0, 2.0, 1.0 or less. In some cases, a sample comprising RNA can have an MN value equal or less than about 8.0. In some cases, a sample comprising RNA can have an RIN value equal or less than about 6.0. In some cases, a sample comprising RNA can have an RIN value equal or less than about 4.0. In some cases, a sample can have an RIN value of less than about 2.0.

A sample, such as an FNA sample, may be obtained from a subject by another individual or entity, such as a healthcare (or medical) professional or robot. A medical professional can include a physician, nurse, medical technician or other. In some cases, a physician may be a specialist, such as an oncologist, surgeon, or endocrinologist. A medical technician may be a specialist, such as a cytologist, phlebotomist, radiologist, pulmonologist or others. A medical professional may obtain a sample from a subject for testing or refer the subject to a testing center or laboratory for the submission of the sample. The medical professional may indicate to the testing center or laboratory the appropriate test or assay to perform on the sample, such as methods of the present disclosure including determining gene sequence data, gene expression levels, sequence variant data, or any combination thereof.

In some cases, a medical professional need not be involved in the initial diagnosis of a disease or the initial sample acquisition. An individual, such as the subject, may alternatively obtain a sample through the use of an over the counter kit. The kit may contain collection unit or device for Obtaining the sample as described herein, a storage unit for storing the sample ahead of sample analysis, and instructions for use of the kit.

A sample can be obtained a) pre-operatively, b) post-operatively, c) after a cancer diagnosis, d) during routine screening following remission or cure of disease, e) when a subject is suspected of having a disease, f) during a routine office visit or clinical screen, g) following the request of a medical professional, or any combination thereof. Multiple samples at separate times can be obtained from the same subject, such as before treatment for a disease commences and after treatment ends, such as monitoring a subject over a time course. Multiple samples can be obtained from a subject at separate times to monitor the absence or presence of disease progression, regression, or remission in the subject.

The sample obtained from the subject may be cytologically ambiguous or suspicious (or indeterminate). In some cases, the sample may be suggestive of the presence of a disease. The volume of sample obtained from the subject may be small, such as about 100 microliters, 50 microliters, 10 microliters, 5 microliters, 1 microliter or less. The sample may comprise a low quantity or quality of polynucleotides, such as a tissue sample with degraded or partially degraded RNA. For example, an FNA sample may yield low quantity or quality of polynucleotides. In such examples, the RNA Integrity Number (RIN) value of the sample may be about 9.0 or less. In some examples, the RIN value may be about 6.0 or less.

### Diseases

A disease, as disclosed herein, can include thyroid cancer. Thyroid cancer can include any subtype of thyroid cancer, including but not limited to, any malignancy of the thyroid gland such as papillary thyroid cancer (PTC), follicular thyroid cancer (FTC), follicular variant of papillary thyroid carcinoma (FVPTC), medullary thyroid carcinoma (MTC), follicular carcinoma (FC), Hurthle cell carcinoma (HC), and/or anaplastic thyroid cancer (ATC). In some cases, the thyroid cancer can be differentiated. In some cases, the thyroid cancer can be undifferentiated.

A thyroid tissue sample can be classified using the methods of the present disclosure as comprising one or more benign or malignant tissue types (e.g., a cancer subtype), including but not limited to follicular adenoma (FA), nodular hyperplasia (NHP), lymphocytic thyroiditis (LCT), and Hurthle cell adenoma (HA), follicular carcinoma (FC), papillary thyroid carcinoma (PTC), follicular variant of papillary carcinoma (FVPTC), medullary thyroid carcinoma (MTC), Mythic cell carcinoma (HC), and anaplastic thyroid carcinoma (ATC), renal carcinoma (RCC), breast carcinoma (BCA), melanoma (MMN), B cell lymphoma (BCL), or parathyroid (PTA).

Other types of cancer of the present disclosure can include but are not limited to adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g., Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g., uterine sarcoma), vaginal cancer, vulvar cancer, or Waldenstrom's macroglobulinemia.

A disease, as disclosed herein, can include hyperproliferative disorders. Malignant hyperproliferative disorders can be stratified into risk groups, such as a low risk group and a medium-to-high risk group. Hyperproliferative disorders can include but are not limited to cancers, hyperplasia, or neoplasia. In some cases, the hyperproliferative cancer can be breast cancer such as a ductal carcinoma in duct tissue of a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, tnyeloproliferative disorders, acute myelogenous leukemia (AML), chronic myelogenous leukemia (CML), mastocytosis, chronic lymphocytic leukemia (CLL), multiple inyeloma (MM), and myelodysplastic, syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer (cancer that begins in the liver); kidney cancer; autoimmune deficiency syndrome (AIDS)-related lymphoma such as diffuse large B-cell lymphoma, B-cell immunoblastic; lymphoma and small non-cleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma, and human papilloma virus (HPV) and cervical cancer; central nervous system (CNS) cancers such as primary brain tumor, which includes gliomas (astrocytoma, anaplastic astrocytoma, or glioblastoma multiforme), oligodendrogliomas, ependymomas, meningiomas, lymphomas, schwannomas, and medulloblastomas; peripheral nervous system (PNS) cancers such as acoustic neuromas and malignant peripheral nerve sheath tumors (MPNST) including neurofibromas and schwannomas, malignant fibrous cytomas, malignant fibrous histiocytomas, malignant meningiomas, malignant mesotheliomas, and malignant mixed Müllerian tumors; oral cavity and oropharyngeal cancer such as hypopharyngeal cancer, laryngeal cancer, nasopharyngeal cancer, and oropharyngeal cancer; stomach cancer such as lymphomas, gastric stromal tumors, and carcinoid tumors; testicular cancer such as genii cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In some cases, the diseases stratified, classified, characterized, or diagnosed by the methods of the present disclosure include but are not limited to thyroid disorders such as for example benign thyroid disorders including but not limited to follicular adenomas, Hurthle cell adenomas, lymphocytic thyroiditis, and thyroid hyperplasia. In some cases, the diseases stratified, classified, characterized, or diagnosed by the methods of the present disclosure include but are not limited to malignant thyroid disorders such as for example follicular carcinomas, follicular variant of papillary thyroid carcinomas, medullary carcinomas, and papillary carcinomas.

Diseases of the present disclosure can include a genetic disorder. A genetic disorder is an illness caused by abnormalities in genes or chromosomes. Genetic disorders can be grouped into two categories: single gene disorders and multifactorial and polygenic (complex) disorders. A single gene disorder can be the result of a single mutated gene. Inheriting a single gene disorder can include but not be limited to autosomal dominant, autosomal recessive, X-linked dominant, X-linked recessive, Y-linked and mitochondrial inheritance. Only one mutated copy of the gene can be necessary for a person to be affected by an autosomal dominant disorder. Examples of autosomal dominant type of disorder can include but are not limited to Huntington's disease, Neurofibromatosis I, Marfan Syndrome, Hereditary nonpolyposis colorectal cancer, or Hereditary multiple exostoses. In autosomal recessive disorders, two copies of the gene must be mutated for a subject to be affected by an autosomal recessive disorder. Examples of this type of disorder can include but are not limited to cystic fibrosis, sickle-cell disease (also partial sickle-cell disease), Tay-Sachs disease, Niemann-Pick disease, or spinal muscular atrophy. X-linked dominant disorders are caused by mutations in genes on the X chromosome such as X-linked hypophosphatemic rickets. Some X-linked dominant conditions such as Rett syndrome, Incontinentia Pigmenti type 2 and Aicardi Syndrome can be fatal. X-linked recessive disorders are also caused by mutations in genes on the X chromosome. Examples of this type of disorder can include but are not limited to Hemophilia A, Duchenne muscular dystrophy, red-green color blindness, muscular dystrophy and Androgenetic alopecia. Y-linked disorders are caused by mutations on the Y chromosome. Examples can include but are not limited to Male Infertility and hypertrichosis pinnae. The genetic disorder of mitochondrial inheritance, also known as maternal inheritance, can apply to genes in mitochondrial DNA such as in Leber's Hereditary Optic Neuropathy.

Genetic disorders may also be complex, multifactorial or polygenic. Polygenic genetic disorders can be associated with the effects of multiple genes in combination with lifestyle and environmental factors. Although complex genetic disorders can cluster in families, they do not have a clear-cut pattern of inheritance. Multifactorial or polygenic, disorders can include heart disease, diabetes, asthma, autism, autoimmune diseases such as multiple sclerosis, cancers, celiopathies, cleft palate, hypertension, inflammatory bowel disease, mental retardation or obesity.

Other genetic disorders can include but are not limited to Ip36 deletion syndrome, 21-hydroxylase deficiency, 22q11.2 deletion syndrome, aceurloplasminemia, achondrogenesis, type H, achondroplasia, acute intermittent porphyria, adenylosuccinate lyase deficiency, Adrenoleukodystrophy, Alexander disease, alkaptonuria, antitrypsin deficiency, Alstrom syndrome, Alzheimer's disease (type 1, 2, 3, and 4), Amelogenesis Imperfecta, amyotrophic lateral sclerosis, Amyotrophic lateral sclerosis type 2, Amyotrophic lateral sclerosis type 4, amyotrophic lateral sclerosis type 4, androgen insensitivity syndrome, Anemia, Angelman syndrome, Apert syndrome, ataxia-telangiectasia, Beare-Stevenson cutis gyrata syndrome, Benjamin syndrome, beta thalassemia, biotinidase deficiency, Birt-Hogg-Dube syndrome, bladder cancer, Bloom syndrome, Bone diseases, breast cancer, Camptomelic dysplasia, Canavan disease, Cancer, Celiac Disease, Chronic Granulomatous Disorder (CGD), Charcot-Marie-Tooth disease, Charcot-Marie-Tooth disease Type 1, Charcot-Marie-Tooth disease Type 4, Charcot-Marie-Tooth disease Type 2, Charcot-Marie-Tooth disease Type 4, Cockayne syndrome, Coffin-Lowry syndrome, collagenopathy types 11 and XI, Colorectal Cancer, Congenital absence of the vas deferens, congenital bilateral absence of vas deferens, congenital diabetes, congenital erythropoietic porphyria, Congenital heart disease, congenital hypothyroidism, Connective tissue disease, Cowden syndrome, Cri du chat syndrome, Crohn's disease, fibrostenosing, Crouzon syndrome, Crouzonodermoskeletal syndrome, cystic fibrosis, De Grouchy Syndrome, Degenerative nerve diseases, Dent's disease, developmental disabilities, DiGeorge syndrome, Distal spinal muscular atrophy type V, Down syndrome, Dwarfism, Ehlers-Danlos syndrome, Ehlers-Danlos syndrome arthrochalasia type, Ehlers-Danlos syndrome classical type, Ehlers-Danlos syndrome dermatosparaxis type, Ehlers-Danlos syndrome kyphoscoliosis type, vascular type, erythropoietic protoporphyria, Fabry's disease, Facial injuries and disorders, factor V Leiden thrombophilia, familial adenomatous polyposis, familial dysautonomia, fanconi anemia, FG syndrome, fragile X syndrome, Friedreich ataxia, Friedreich's ataxia, G6PD deficiency, galactosemia, Gaucher's disease (type 1, 2, and 3), Genetic brain disorders, Glycine encephalopathy, Haemochromatosis type 2, Haemochromatosis type 4, Harlequin Ichthyosis, Head and brain malformations, Hearing disorders and deafness, Hearing problems in children, hemochromatosis (neonatal, type 2 and type 3), hemophilia, hepatoerythropoietic porphyria, hereditary coproporphyria, Hereditary Multiple Exostoses, hereditary neuropathy with liability to pressure palsies, hereditary nonpolyposis colorectal cancer, homocystinuria, Huntington's disease, Hutchinson Gilford Progeria. Syndrome, hyperoxaluria, primary, hyperphenylalaninemia, hypochondrogenesis, hypochondroplasia, idic15, incontinentia pigmenti, Infantile Gaucher disease, infantile-onset ascending hereditary spastic paralysis, Infertility, Jackson-Weiss syndrome, Joubert syndrome, Juvenile Primary Lateral Sclerosis, Kennedy disease, Klinefelter syndrome, Kniest dysplasia, Krabbe disease, Learning disability, Lesch-Nyhan syndrome, Leukodystrophies, Li-Fraumeni syndrome, lipoprotein lipase deficiency, familial, Male genital disorders, Marfan syndrome, McCune-Albright syndrome, McLeod syndrome, Mediterranean fever, familial, Menkes disease, Menkes syndrome, Metabolic disorders, methemoglobinemia beta-globin type, Methemoglobinemia congenital methaernoglobinaemia, methylmalonic acidemia, Micro syndrome, Microcephaly, Movement disorders, Mowat-Wilson syndrome, Mucopolysaccharidosis (MPS I), Muenke syndrome, Muscular dystrophy, Muscular dystrophy, Duchenne and Becker type, muscular dystrophy, Duchenne and Becker types, myotonic dystrophy, Myotonic dystrophy type I and type 2, Neonatal hemochromatosis, neurofibromatosis, neurofibromatosis neurofibromatosis 2, Neurofibromatosis type I, neurofibromatosis type II, Neurologic diseases, Neuromuscular disorders, Niemann-Pick disease, Nonketotic hyperglycinemia, nonsyndromic deafness, Nonsyndromic deafness autosomal recessive, Noonan syndrome, osteogenesis imperfecta (type I and type otospondylomegaepiphyseal dysplasia, pantothenate kinase-associated neurodegeneration, Patau Syndrome (Trisomy 13), Pendred syndrome, Peutz-Jeghers syndrome, Pfeiffer syndrome, phenylketonuria, porphyria, porphyria cutanea tarda, Prader-Willi syndrome, primary pulmonary hypertension, prion disease, Progeria, propionic acidemia, protein C deficiency, protein S deficiency, pseudo-Gaucher disease, pseudoxanthoma elasticum, Retinal disorders, retinoblastoma, retinoblastoma LA Friedreich ataxia, Rett syndrome, Rubinstein-Taybi syndrome, Sandhoff disease, sensory and autonomic neuropathy type III, sickle cell anemia, skeletal muscle regeneration, Skin pigmentation disorders, Smith Lemli Opitz Syndrome, Speech and communication disorders, spinal muscular atrophy, spinal-bulbar muscular atrophy, spinocerebellar ataxia, spondyloepimetaphyseal dysplasia, Strudwick type, spondyloepiphyseal dysplasia congenita, Stickler syndrome, Stickler syndrome COL2A Tay-Sachs disease, tetrahydrobiopterin deficiency, thanatophoric dysplasia, thiamine-responsive megaloblastic anemia with diabetes mellitus and sensorineural deafness, Thyroid disease, Tourette's Syndrome, Treacher Collins syndrome, triple X syndrome, tuberous sclerosis, Turner syndrome, Usher syndrome, variegate porphyria, von Hippel-Lindau disease, Waardenburg syndrome, Weissenbacher-Zweymüller syndrome, Wilson disease, Wolf-Hirschhorn syndrome, Xerodenna Pigmentosum, X-linked severe combined immunodeficiency, X-linked sideroblastic anemia, or X-linked spinal-bulbar muscle atrophy.

### Cytological Analysis

The methods and systems as described herein, including processing a biological sample may include cytological analysis of samples. Examples of cytological analysis include cell staining techniques and/or microscope examination performed by any number of methods and suitable reagents including but not limited to: eosin-azure (EA) stains, hematoxylin stains, CYTO-STAIN™, papanicolaou stain, eosin, nissl stain, toluidine blue, silver stain, azocarmine stain, neutral red, or janus green. More than one stain can be used in combination with other stains. In some cases, cells are not stained at all. Cells can be fixed and/or permeabilized with for example methanol, ethanol, glutaraldehyde or formaldehyde prior to or during the staining procedure. In some cases, the cells may not be fixed. Staining procedures can also be utilized to measure the nucleic acid content of a sample, for example with ethidium bromide, hematoxylin, nissl stain or any other nucleic acid stain.

Microscope examination of cells in a sample can include smearing cells onto a slide by standard methods for cytological examination. Liquid based cytology (LBC) methods may be utilized. In some cases, LBC methods provide for an improved approach of cytology slide preparation, more homogenous samples, increased sensitivity and specificity, or improved efficiency of handling of samples, or any combination thereof. In LBC methods, samples can be transferred from the subject to a container or vial containing a LBC preparation solution such as for example CYTYC THINPREP®, SUREPATH™, or MONOPREP® or any other LBC preparation solution. Additionally, the sample may be rinsed from the collection device with LBC preparation solution into the container or vial to ensure substantially quantitative transfer of the sample. The solution containing the sample in LBC preparation solution may then be stored and/or processed by a machine or by one skilled in the art to produce a layer of cells on a glass slide. The sample may further be stained and examined under the microscope in the same way as a conventional cytological preparation.

Samples can be analyzed by immuno-histochemical staining. Immuno-histochemical staining can provide analysis of the presence, location, and distribution of specific molecules or antigens by use of antibodies in a sample (e.g., cells or tissues). Antigens can be small molecules, proteins, peptides, nucleic acids or any other molecule capable of being specifically recognized by an antibody. Samples may be analyzed by immuno-histochemical methods with or without a prior fixing and/or permeabilization step. In some cases, the antigen of interest may be detected by contacting the sample with an antibody specific for the antigen and then non-specific binding may be removed by one or more washes. The specifically bound antibodies may then be detected by an antibody detection reagent such as for example a labeled secondary antibody, or a labeled avidin/streptavidin. The antigen specific antibody can be labeled directly. Suitable labels for immuno-histochemistry include but are not limited to fluorophores such as fluorescein and rhodamine, enzymes such as alkaline phosphatase and horse radish peroxidase, or radionuclides such as ³²P and ¹²⁵I. Gene product markers that may be detected by immuno-histochemical staining include but are not limited to Her2/Neu, Ras, Rho, EGFR, VEGFR, UbcH10, RET/PTC1, cytokeratin 20, calcitonin, GAL-3, thyroid peroxidase, or thyroglobulin.

Metrics associated with assigning a noise-call to a gene of a biological dataset as disclosed herein, such as gene expression levels or sequence variant information or fusions, need not be a characteristic of every cell of a sample. Thus, the methods disclosed herein can be useful for modifying a biological dataset where less than all cells within the sample exhibit a complete pattern of the gene expression levels or sequence variant information, fusions, or other data indicative of a noise-call.

Routine cytological or other assays may indicate a sample as negative (without disease), diagnostic (positive diagnosis for disease, such as cancer), ambiguous or suspicious (suggestive of the presence of a disease, such as cancer), or non-diagnostic (providing inadequate information concerning the presence or absence of disease). The methods as described herein may confirm results from the routine cytological assessments or may provide an original assessment similar to a routine cytological assessment in the absence of one. The methods as described herein may classify a sample as malignant or benign, including samples found to be ambiguous or suspicious.

### Classification

A sample can be classified as positive or negative for a disease with an accuracy of at least about 50%, 60%, 70%, 75%, 80%, 85%, 90%, 95%, 96%, 97%, 98%, 99% or more. A sample can be classified with an accuracy of at least about 70%. A sample can be classified with an accuracy of at least about 80%. A sample can be classified with an accuracy of at least about 85%. A sample can be classified with an accuracy of at least about 90%. A sample can be classified with an accuracy of at least about 91%. A sample can be classified with an accuracy of at least about 92%. A sample can be classified with an accuracy of at least about 93%. A sample can be classified with an accuracy of at least about 94%. A sample can be classified with an accuracy of at least about 95%. A sample can be classified with an accuracy of at least about 96%. A sample can be classified with an accuracy of at least about 97%. A sample can be classified with an accuracy of at least about 98%. A sample can be classified with an accuracy of at least about 99%. A sample can be classified as benign, malignant, or non-diagnostic with an accuracy of greater than about 50%, 60%, 70%, 75%, 80%, 85%, 90%, 95%, 96%, 97%, 98%, 99% or more. Accuracy can be calculated using a classifier.

A sample can be classified as positive or negative for a disease with a specificity of at least about 50%, 60%, 70%, 75%, 80%, 85%, 90%, 95%, 96%, 97%, 98%, 99% or more. A sample can be classified with a specificity of at least about 70%. A sample can be classified with a specificity of at least about 80%. A sample can be classified with a specificity of at least about 85%. A sample can be classified with a specificity of at least about 90%. A sample can be classified with a specificity of at least about 91%. A sample can be classified with a specificity of at least about 92%. A sample can be classified with a specificity of at least about 93%. A sample can be classified with a specificity of at least about 94%. A sample can be classified with a specificity of at least about 95%. A sample can be classified with a specificity of at least about 96%. A sample can be classified with a specificity of at least about 97%. A sample can be classified with a specificity of at least about 98%. A sample can be classified with a specificity of at least about 99%. A sample can be classified as benign, malignant, or non-diagnostic with a specificity of greater than about 50%, 60%, 70%, 75%, 80%, 85%, 90%, 95%. 96%, 97%, 98%, 99% or more. Specificity can be calculated using a classifier.

A sample can be classified as positive or negative for a disease with a sensitivity of at least about 50%, 60%, 70%, 75%, 80%, 854% 90%, 95%, 96%, 97%, 98%, 99% or more. A sample can be classified with a sensitivity of at least about 70%. A sample can be classified with a sensitivity of at least about 80%. A sample can be classified with a sensitivity of at least about 85%. A sample can be classified with a sensitivity of at least about 90%. A sample can be classified with a sensitivity of at least about 91%. A sample can be classified with a sensitivity of at least about 92%. A sample can be classified with a sensitivity of at least about 93%. A sample can be classified with a sensitivity of at least about 94%. A sample can be classified with a sensitivity of at least about 95%. A sample can be classified with a sensitivity of at least about 96%. A sample can be classified with a sensitivity of at least about 97%. A sample can be classified with a sensitivity of at least about 98%. A sample can be classified with a sensitivity of at least about 99%. A sample can be classified as benign, malignant, or non-diagnostic with a sensitivity of greater than about 50%, 60%, 70%, 75%, 80%, 85%, 90%, 95%, 96%, 97%, 98%, 99% or more. Sensitivity can be calculated using a classifier.

Methods and systems as described herein for classifying samples as benign, malignant, or non-diagnostic can have a positive predictive value of at least about 55%, 60%, 65%, 70%, 75%, 80%, 85%, 90%, 95?% 95.5%, 96%, 96.5%, 97%, 97.5%, 98%, 98.5%, 99%, 99.5% or more; and/or a negative predictive value of at least about 55%, 60%, 65%, 70%, 75%, 80%, 85%, 90%, 95%, 95.5%, 96%, 96.5%, 97%, 97.5%, 98%, 98.5%, 99%, 99.5% or more. Positive predictive value (PPV), or precision rate, or post-test probability of disease, can be the proportion of subjects with positive test results who are correctly diagnosed or correctly stratified into risk groups. It can be an important measure because it can reflect the probability that a positive test reflects the underlying disease being tested for its value can depend on the prevalence of the disease, which may vary. The negative predictive value (NPV) can be the proportion of subjects with negative test results who are correctly diagnosed. PPV and NPV measurements can be derived using appropriate disease subtype prevalence estimates. For subtype specific estimates, disease prevalence may sometimes be incalculable because there may not be any available samples.

The classifier or trained algorithm of the present disclosure can be used to classify a modified biological sample as positive or negative for a disease, such as cancer. The classifier may classify the biological sample as benign or malignant for cancer. One or more selected feature spaces such as gene expression level and sequence variant data can be provided alone or in combination to a classifier or trained algorithm. The feature space may be a modified feature space. Illustrative algorithms can include but are not limited to methods that reduce the number of variables such as a principal component analysis algorithm, partial least squares method, or independent component analysis algorithm. Illustrative algorithms can include methods that handle large numbers of variables directly such as statistical methods or methods based on machine learning techniques. Statistical methods can include penalized logistic regression, prediction analysis of microarrays (PAM), methods based on shrunken centroids, support vector machine analysis, or regularized linear discriminant analysis. Machine learning techniques can include bagging procedures, boosting procedures, random forest algorithms, or any combination thereof.

Genome-wide RNA Sequence (RNASeq) data (80 million reads per sample) may be obtained and supervised learning may be used to train classifiers. Training of classifiers may include a Support Vector Machine (SVM) model, a Random Forest (RF) model, a Least Absolute Shrinkage and Selection Operator (LASSO) model, an Ensemble 1 model, a Penalized Logistic Regression (PLR) model, or any combination thereof. Classifier performance may be measured using 10-fold cross-validation on the same sample cohort. Classifiers may be built using one or more genes (such as one or more genes that are not blacklisted, such as 10, 50, 100, 150, 200, 250, 300 genes or more) and open source software DESeq models.

The classifier or trained algorithm of the present disclosure can comprise two or more feature spaces. The two or more feature spaces can be unique or distinct from one another. Individual feature spaces can comprise types of information about a sample, such as gene expression level data, sequence variant data, or fusions. Combining two or more feature spaces in a classifier can produce a higher level of accuracy of the classifying than using a single feature space. The dynamic ranges of the individual feature spaces can be different, such as at least 1 or 2 orders of magnitude different. For example, the dynamic range of the gene expression level feature space may be between 0 and about 300 and the dynamic range of sequence variant feature space may be between 0 and about 20.

Individual feature spaces can comprise a set of genes, such as a first set of genes of the first feature space and a second set of genes of the second feature space. A set of genes of an individual feature space can be associated with a disease, such as cancer. The first set of genes and the second set of genes can be the same set. The first set of genes and the second set of genes can be different sets. The first set of genes or the second set of genes can comprise less than about 1000, 500, 400, 300, 200, 100, 75, 70, 65, 60, 55, 50, 45, 40, 35, 30, 25, 20, 15, 10, 5 genes or less. The first set of genes or the second set of genes can comprise less than about 10 genes. The first set of genes or the second set of genes can comprise less than about 50 genes. The first set of genes or the second set of genes can comprise less than about 75 genes. The first set of genes or the second set of genes can comprise between about 50 and about 400 genes. The first set of genes or the second set of genes can comprise between about 50 and about 200 genes. The first set of genes or the second set of genes can comprise between about 10 and about 600 genes. The first set of genes may comprise a modified set of genes. The second set of genes may comprise a modified set of genes.

The classifier or trained algorithm of the present disclosure can be trained using a set of samples, such as a training set or sample cohort. The sample cohort can comprise about 5, 10, 20, 30, 40, 50, 60, 70, 80, 90, 100, 150, 200, 250, 300, 350, 400, 450, 500, 600, 700, 800, 900, 1000, 2000, 3000, 4000, 5000 or more independent samples. The sample cohort can comprise about 100 independent samples. The sample cohort can comprise less than about 100 independent samples. The sample cohort can comprise less than about 50 independent samples. The sample cohort can comprise less than about 30 independent samples. The sample cohort can comprise about 200 independent samples. The sample cohort can comprise between about 100 and about 500 independent samples. The independent samples can be from subjects having been diagnosed with a disease, such as cancer, from healthy subjects, or any combination thereof.

The sample cohort or training set can comprise samples from about 5, 10, 20, 30, 40, 50, 60, 70, 80, 90, 100, 150, 200, 250, 300, 350, 400, 450, 500, 600, 700, 800, 900, 1000 or more different individuals. The sample cohort can comprise samples from about 100 different individuals. The sample cohort can comprise samples from about 200 different individuals. The different individuals can be individuals having been diagnosed with a disease, such as cancer, health individuals, or any combination thereof.

The sample cohort can comprise samples obtained from individuals living in at least 1, 2, 3, 4, 5, 6, 67, 8, 9, 10, 15, 20, 25, 30, 35, 40, 45, 50, 55, 60, 65, 70, 75, or 80 different geographical locations (e.g., sites spread out across a nation, such as the United States, across a continent, or across the world). Geographical locations include, but are not limited to, test centers, medical facilities, medical offices, post office addresses, cities, counties, states, nations, or continents. In some cases, a classifier that is trained using sample cohorts from the United States may need to be re-trained for use on sample cohorts from other geographical regions (e.g., India, Asia, Europe, Africa, etc.).

A classifier or trained algorithm may produce a unique output each time it is run. For example, using different samples with the same classifier can produce a unique output each time the classifier is run. Using the same samples with the same classifier can produce a unique output each time the classifier is run. Using the same samples to train a classifier more than one time, may result in unique outputs each time the classifier is run.

Data from the methods described, such as gene expression levels, fusions, or sequence variant data can be further analyzed using feature selection techniques such as filters which can assess the relevance of specific features by looking at the intrinsic properties of the data, wrappers which embed the model hypothesis within a feature subset search, or embedded protocols in which the search for an optimal set of features is built into a classifier algorithm.

Filters useful in the methods of the present disclosure can include (1) parametric methods such as the use of two sample t-tests, analysis of variance (ANOVA) analyses, Bayesian frameworks, or Gamma distribution models (2) model free methods such as the use of Wilcoxon rank sum tests, between-within class sum of squares tests, rank products methods, random permutation methods, or threshold number of misclassification (TNoM) which involves setting a threshold point for fold-change differences in expression between two datasets and then detecting the threshold point in each gene that minimizes the number of mis-classifications or (3) multivariate methods such as bivariate methods, correlation based feature selection methods (CFS), minimum redundancy maximum relevance methods (MRMR), Markov blanket filter methods, and uncorrelated shrunken centroid methods. Wrappers useful in the methods of the present disclosure can include sequential search methods, genetic algorithms, or estimation of distribution algorithms. Embedded protocols can include random forest algorithms, weight vector of support vector machine algorithms, or weights of logistic regression algorithms.

Statistical evaluation of the results obtained from the methods and systems described herein can provide a quantitative value or values indicative of one or more of the following: classification of a sample as positive or negative for a disease such as cancer. Thus a medical professional, who may not be trained in genetics or molecular biology, need not understand gene expression level or sequence variant data results. Rather, data can be presented directly to the medical professional in its most useful form to guide care or treatment of the subject. Statistical evaluation, combination of separate data results, and reporting useful results can be performed by a classifier or trained algorithm. Statistical evaluation of results can be performed using a number of methods including, but not limited to: the students T test, the two sided. T test, Pearson rank sum analysis, hidden markov model analysis, analysis of q-q plots, principal component analysis, one way analysis of variance (ANOVA), two way ANOVA, and the like. Statistical evaluation can be performed by the classifier or trained algorithm. In some cases, such quantitative value or values do not directly yield a diagnosis, but may be used by a healthcare professional (e.g., physician) to diagnose a subject.

Methods and systems of the present disclosure may enable a subject to be treated for a disease. This may include provide the subject or another user (e.g., healthcare provider) with a therapeutic intervention, such as a report indicative of the quantitative value or values, or a statistical evaluation of results of an assay performed on a biological sample of the subject. Such therapeutic intervention may including providing a recommended treatment to the subject or the user, or treating the subject (e.g., administering a drug to treat thyroid cancer or removing at least a portion of the thyroid of the subject). In some examples, methods and systems of the present disclosure enable a subject to be treated for cancer, such as thyroid or lung cancer.

The methods and systems disclosed herein may include extracting and analyzing protein or nucleic acid (RNA or DNA) from one or more samples from a subject. Nucleic acid can be extracted from the entire sample obtained or can be extracted from a portion. In some cases, the portion of the sample not subjected to nucleic acid extraction may be analyzed by cytological examination or immuno-histochemistry. Methods for RNA or DNA extraction from biological samples can include for example phenol-chloroform extraction (such as guanidinium thiocyanate phenol-chloroform extraction), ethanol precipitation, spin column-based purification, or others.

General methods for determining gene expression levels may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immuno-absorbance assays, mass-spectrometry, immuno-histochemistry, blotting, sequencing, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Gene expression product levels may be normalized to an internal standard such as total messenger ribonucleic acid (mRNA) or the expression level of a particular gene. There can be a specific difference or range of difference in gene expression between samples being compared to one another, for example a sample from a subject and a reference sample. The difference in gene expression level can be at least 5%, 10%, 15%, 20%, 25%, 30%, 35%, 40%, 45% or 50% or more. In some cases, the difference in gene expression level can be at least 2, 3, 4, 5, 6, 7, 8, 9, 10 fold or more.

RNA Sequencing can produce two or more feature spaces such as counts of gene expression and presence of sequence variants or fusions of a particular sample. For example, RNA sequencing measures variants in genes expressed in a specific tissue or specific sample, such as a thyroid tissue or thyroid nodule. Next generation sequence can provide gene expression level data of a particular sample. Sequencing results, such as RNA sequencing and Next generation sequencing results, can be entered into a classifier that can combine unique feature spaces to determine the presence or absence of a disease in a biological sample. The classifier or trained algorithm can include algorithms that have been developed using a modified biological dataset.

### Markers for Array Hybridization, Sequencing, Amplification

Suitable reagents for conducting array hybridization, nucleic acid sequencing, nucleic acid amplification or other amplification reactions include, but are not limited to, DNA polymerases, markers such as forward and reverse primers, deoxynucleotide triphosphates (dNTPs), and one or more buffers. Such reagents can include a primer that is selected for a given sequence of interest, such as the one or more genes of the first set of genes and/or second set of genes.

In such amplification reactions, one primer of a primer pair can be a forward primer complementary to a sequence of a target polynucleotide molecule (e.g., the one or more genes of the first or second sets) and one primer of a primer pair can be a reverse primer complementary to a second sequence of the target polynucleotide molecule and a target locus can reside between the first sequence and the second sequence.

The length of the forward primer and the reverse primer can depend on the sequence of the target polynucleotide (e.g., the one or more genes of the first or second sets) and the target locus. In some cases, a primer can be greater than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, or about 100 nucleotides in length. As an alternative, a primer can be less than about 100, 95, 90, 85, 80, 75, 70, 65, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or about nucleotides in length. In some cases, a primer can be about 15 to about 20, about 15 to about 25, about 15 to about 30, about 15 to about 40, about 15 to about 45, about 15 to about 50, about 15 to about 55, about 15 to about 60, about 20 to about 25, about 20 to about 30, about 20 to about 35, about 20 to about 40, about 20 to about 45, about 20 to about 50, about 20 to about 55, about 20 to about 60, about 20 to about 80, or about 20 to about 100 nucleotides in length.

Primers can be designed according to known parameters for avoiding secondary structures and self-hybridization, such as primer dimer pairs. Different primer pairs can anneal and melt at about the same temperatures, for example, within 1° C., 2° C., 3° C., 4° C., 5° C., 6° C., 7° C., 8° C., 9° C. or 10° C. of another primer pair.

The target locus can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends or 5′ ends of the plurality of template polynucleotides.

The markers (i.e., primers) for the methods described can be one or more of the same primer. In some instances, the markers can be one or more different primers such as about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 or more different primers. In such examples, each primer of the one or more primers can comprise a different target or template specific region or sequence, such as the one or more genes of the first or second sets.

The one or more primers can comprise a fixed panel of primers. The one or more primers can comprise at least one or more custom primers. The one or more primers can comprise at least one or more control primers. The one or more primers can comprise at least one or more housekeeping gene primers. In some instances, the one or more custom primers anneal to a target specific region or complements thereof. The one or more primers can be designed to amplify or to perform primer extension, reverse transcription, linear extension, non-exponential amplification, exponential amplification, PCR, or any other amplification method of one or more target or template polynucleotides.

Primers can incorporate additional features that allow for the detection or immobilization of the primer but do not alter a basic property of the primer (e.g., acting as a point of initiation of DNA synthesis). For example, primers can comprise a nucleic acid sequence at the 5′ end which does not hybridize to a target nucleic acid, but which facilitates cloning or further amplification, or sequencing of an amplified product. For example, the sequence can comprise a primer binding site, such as a PCR priming sequence, a sample barcode sequence, or a universal primer binding site or others.

A universal primer binding site or sequence can attach a universal primer to a polynucleotide and/or amplicon. Universal primers can include—47F (M13F), alfaMF, AOX3′, AOX5′, BGHr, CMV-30, CMV-50, CVMf, LACrmt, lamgda gt10F, lambda gt 10R, lambda gt11F, lambda gt11R, M13 rev, M13Forward(−20), M13Reverse, male, p10SEQPpQE, pA-120, pet4, pGAP Forward, pGLRVpr3, pGLpr2R, pKLAC14, pQEFS, pQERS, pucU1, pucU2, reversA, seqIREStam, seqIRESzpet, seqori, seqPCR, seqpIRES-, seqpIRES+, seqpSecTag, seqpSecTag+, seqretro+PSI, SP6, T3-prom, T7-prom, and T7-termInv. As used herein, attach can refer to both or either covalent interactions and noncovalent interactions. Attachment of the universal primer to the universal primer binding site may be used for amplification, detection, and/or sequencing of the polynucleotide and/or amplicon.

### Kits

The disease diagnostic business, molecular profiling business, pharmaceutical business, or other business associated with patient healthcare may provide a kit for performing the processing a biological sample. The kit may include a classifier, a sample cohort or training set for training the algorithm, and a list of genes for each feature space, such as genes having a high signal to noise ratio. In some cases, the kit may include a classifier and a list of genes for each feature space. The kit may be a general kit for all disease types. The kit may be a specific kit for a specific disease such as cancer, or a specific kit to a disease subtype such as thyroid cancer. The kit may provide a classifier that has already been trained used a sample cohort or training set not provided in the kit. The kit may provide periodic updates of sample cohorts or lists of genes for feature spaces to use or not to use with the classifier. The kit may provide software to automate a summary of results that can be reported or displayed or downloaded by the medical professional and/or entered into a database, such as genes that may be flagged or blacklisted. The summary of results can include any of the results disclosed herein, including recommendations of treatment options for the patient and risk occurrence of a disease. The kit may also provide a unit or device for obtaining a sample from a subject (e.g., a device with a needle coupled to an aspirator). The kit may also provide instructions for performing methods as disclosed herein, and include all necessary buffers and reagents for RNA sequencing and next generation (NextGen) sequencing. The kit may also include instructions for analyzing the results. Such instructions may include directing the user to software (e.g., software with a trained algorithm) and databases for analyzing the results.

### Computer Control Systems

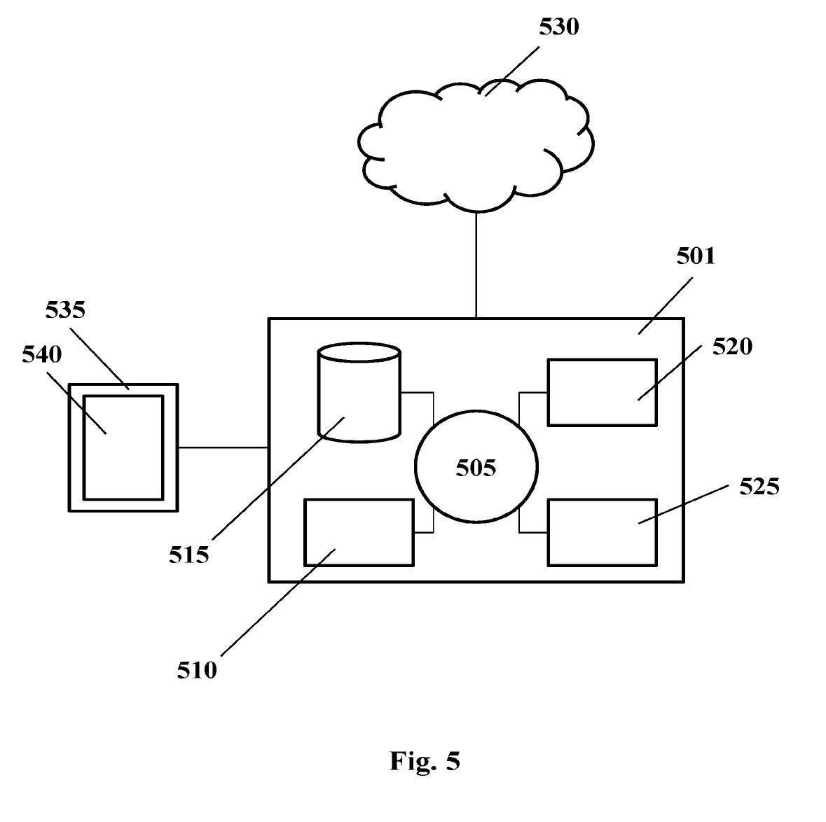

The present disclosure provides computer control systems that are programmed to implement methods of the disclosure. FIG. 5 shows a computer system 501 that is programmed or otherwise configured to implement the methods provided herein. The computer system 501 can regulate various aspects of the train algorithm, the filtering of sample types, the analysis of gene expression levels, sequence variant information and others of the present disclosure, such as, for example, comparing gene expression levels between a biological sample and a control sample. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, an output or readout of the trained algorithm. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, determine whether a biological tissue is malignant or benign for a cancer with a high degree of accuracy, such as about 90%.

### Example 1

Classifier scores obtained for a particular sample(s) may bias systematically as a function of the reagent lot(s) used to process that sample(s). Such systemic bias may be characterized as a deviation model derived from running a set of training/reference samples using a particular combination of critical reagent lots and comparing those scores to original ‘reference’ scores (i.e., scores linked to diagnostic truth).

For RNASeq, a variant call may be made based on whether a particular nucleotide is the reference allele or a variant, or whether a fusion gene is being expressed. In the case of RNASeq, a call can be made for a pre-specified panel of variants and fusion or for the entire genome. The ability to make such calls largely depends on the number of reads available for each gene being measured, which may vary in a reagent lot-dependent way. However, because the call is dependent on the presence or absence of reads, it cannot be corrected algorithmically. Therefore, this study focuses on identifying variation as it pertains to the ability of the software pipeline to detect variants and fusions across different reagent lots. A few of the control samples may be expected to contain many variants at different levels (UHR) or relatively few variants (NA12878). FNA samples may be selected that contain different variants based on exome or Ampli Seq (Ion Torrent) DNA sequencing reference data. As an outcome from this study, variants (especially variants pre-specified on a panel) lacking consistency across reagent lots may be defined as undesirable for future commercial products (i.e., ‘black list’ for variants). Note that variant calls may depend on the expression level (detected as read depth, e.g., minimum read depth of 10 to make a call), which indicates that the consistency of variant calls can be different as expression levels change across different samples and sample types. This may be considered in the black list evaluation.

For gene expression level analysis using RNASeq, this study may provide the initial data for the evaluation of the gene level total reproducibility across reagent lots, operators, sample types, runs, and instruments. Specifically for classifier training, this may aid in determining which genes to potentially avoid to make a classifier more robust to systemic or extreme random variations (such as reagent originated variations) and in turn avoid potential reagent calibration needs. Such genes may still be valuable to certain classifiers, and such variations of individual genes may be likely linked to expression levels and sample types (i.e., may empirically generate different “black lists” for different products). The intention of the black list may be to inform a classifier training but not to exclude the blacklisted genes preemptively.

Additionally, when both RNASeq and an existing microarray based gene expression classifier are available, a secondary classifier score may be generated based on the RNASeq expression levels of a number of genes (variants, and genomic loss-of-heterozygozity (LOH) determinations may also contribute as features used during training of the classifiers). The derived score may be subject to variation as a function of reagent lot. This study provides the data that can be used to further estimate the magnitude of such an effect through the use of thyroid FNA samples as well as lung TBB samples, when the corresponding classifiers are available.

The FNA samples may be selected to represent a variety of sub-types of thyroid cancer to try to maximize the expression level differences among this cohort of samples. The TBB samples may similarly be selected to represent a range of UIP/non-UIP scores and RNA quality.

### Materials

Materials: The samples used in this study are outlined below in the study design section and are listed again in the results. Reagents: Multiple manufacturing lots of all critical reagents susceptible to batch effects (i.e., RNA Access kit and its individual components) that are used are outlined below in the study design section. Software: An analysis pipeline is used to process data from raw sequencing output to gene expression levels, to variant and fusion calling. In this study, splice-aware alignments are generated using reference genome 37 and STAR v2.4.1b, and Ensemble 75. De-duplication used Picard v1.123 and MarkDuplicates. Read processing uses GATK v.3.3 and the GATK Haplotype caller, and fusion detection uses Chimera software.

### Study Parameters

Sample selection: Eight control samples are included in this study, each run in triplicates on each of three reagent lots:

1) The Universal Human Reference (UHR; Agilent)—Because the UHR sample is a mix of 10 different cell lines, it is likely that some variants are present at a low frequency, potentially as low as 5%. For example, if one of the component cell lines contains a heterozygous allele at a position of a common germline SNP (50% allele frequency in that cell line) and the other component cell lines are all homozygous reference at that same position, the final allele frequency at that position may be 5%. The UHR sample therefore represents an opportunity to identify low frequency variants in a control sample. The final frequency that is detected in the RNA is unknown due to potential variation in the level of expression between each component cell line.

2) NA12878 total RNA (manufactured at Microbiology & Quality Associates); Per the data released by the Genome in a Bottle Consortium, NA12878 contains 117 variants that are contained within the amplicons comprising the pre-specified AmpliSeq 851 panel. Of these 117 variants, 67 are heterozygous, and 50 are homozygous.

3) M-RNA-005, which has been analyzed by RNASeq multiple times;

4) B-RNA-004;

5) Human Thyroid Total RNA (Thyroid-636536; Clontech), which is made from a pool of 65 thyroid tissues;

6) Human Lung Tumor Total RNA (LT-636633; Clontech);

7) Human Lung Control RNA (LC-RNA; Agilent); and

8) Human Brain Reference Total RNA (Brain-AM6050; LifeTechnologies), which is pooled from multiple donors and several brain regions.

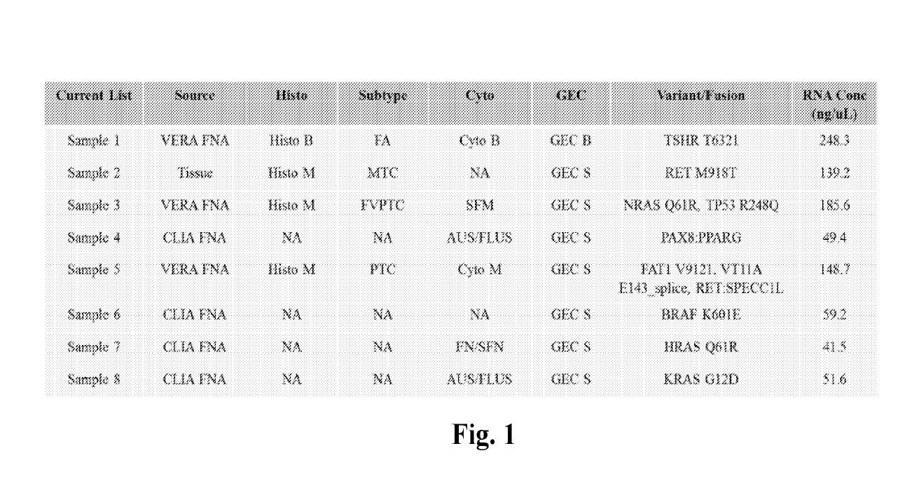

The eight thyroid FNA samples that are used in this effort are run in duplicate on each of three reagent lots. The list of FNA samples are shown in FIG. 1. In addition, because the current focus is on variant and fusion detection for Afirma Plus Phase I, samples are selected based on the following criteria:

A) A minimum of 90 ng total RNA available;

B) Samples are selected if they have a variant or fusion in a high-value thyroid cancer-related gene such as BRAF, HRAS, KRAS, NRAS, TSHR, or RET. Existing exome, AmpliSeq DNA, or RNA Access data is used to assess the presence of variants, and existing RNA Access or high read depth NuGen Ovation v2 RNA sequencing is used to assess the presence of fusions; and

C) As much as possible, the FNA samples are selected to represent different thyroid cancer sub-types.

The eight TBB samples are run a single time on each reagent lot. The criteria for TBB sample selection from among TBBs extracted during project feasibility are as follows:

A) Estimated RNA mass remaining >200 ng;

B) Sample-level pathology current truth of CIF, NOC no preference, Other, NA, or Non-diagnostic (eg. samples lacking pathology truth useful in training or in scoring classifier performance); and

C) Samples processed via the optimized microarray assay and scored using the 300-feature GLMnet U1P/non-UIP classifier.

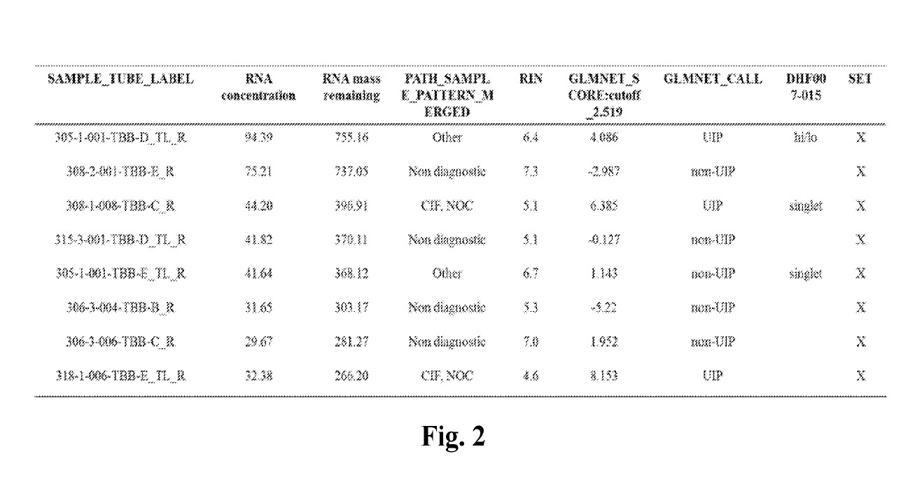

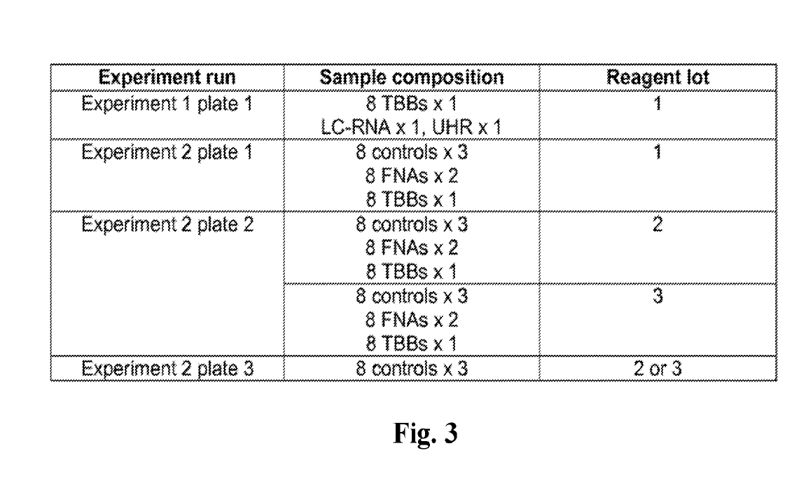

16 samples were selected which span a range of U1P/non-UIP classification scores and RNA RINs. These samples were sub-grouped into two sets of 8 (Sets A and B) such that samples from the same patient were distributed as much as possible between the two sets. Set B was defined for use in the current study, and contains 3 samples processed manually as part of the RNA Access TBB Feasibility study, as shown in FIG. 2. The samples to be processed are composed of 24 unique biological samples. Two processing runs are performed utilizing 3 different lots of the RNA Access library preparation kit (one run of 96 samples total use two separate reagent lots). These samples may be processed alongside other samples to allow for more efficient library preparation on the automated platform. Additionally, one more run is processed with only the 8 control samples in triplicates, utilizing one of the 3 reagent lots used in the first three runs. The last run contributes to the model to partially separate run effects from reagent lot effects (see the mixed effect model in Appendix). Additionally, the same 8 TBB samples and two of the control samples (LC-RNA and UHR) were also included in both experiment 1 and 2 using a single reagent lot. These samples can also contribute to the model. FIG. 3 shows the design and data available to use.

### Quality Control

In process, quality control (QC) metrics are evaluated against the following criteria:

A) The polymerase chain reaction (PCR)1 concentration of each sample must be >20 ng/uL;

B) The PCR2 concentration of each pool of samples must be >15 nM;

C) Sequencing QC metrics are evaluated against the following criteria;

D) A minimum of 10 million reads from each sample;

E) Less than 80% duplicate reads;

F) A minimum of 60% reads mapping to exonic regions.

Samples that fail any of the above QC criteria may not be included in the final data analysis but may continue to be processed through the entire assay. Any run that fails any QC metrics defined in section 5.2 or underperforms with known or clearly traceable root causes is allowed to be repeated.

### Data Analysis

Primary analysis results (alignment (BAM), variant calls (VCF), fusion calls) from a total number of 48×3 test samples (from 24 unique biological samples) are available for Data Analysis. In the following analysis description, ‘Run’ is a short term for experiment run (i.e., plate) and ‘Lot’ is a short term for reagent lot.

Variant Calling:

If necessary, the variant calls from primary analysis are to be further processed to meet the final calling criteria, as defined additional studies. At each marker, outcome from each assay is one of three: (1) no-call (2) reference or (3) variant call. If criteria to define ‘no-call’ are not finalized at the time of evaluation, the exercise below can be repeated with a few choices of such criteria starting with the default GATK pipeline implemented currently. Evaluation is focused on a pre-specified 851 variant panel. In the future and if time allows, similar analysis maybe extended to larger panels, such as markers with known variants in the control samples. Run and reagent lot effects are evaluated at two levels: (1) marker-level and (2) panel-level (i.e., on a pre-defined set of markers as a whole). Marker-level evaluation informs existence of any markers to be excluded prior to panel-level commercial product performance evaluation. Panel-level evaluation summarizes overall magnitude of run/lot effect.

Read Depth Evaluation:

Total # of reads (at marker) is essential information in separating no-call from reference call or variant call. In particular, variability in relatively low read count (0-30× range) as a function of run/lot-to-run/lot variability (and reagent lot in particular) is of great interest. Variability can be explored in multiple scales: (1) raw count (2) normalized count (e.g., log scale) and (3) ordered bins focusing on low read count (0, >3, >5, >8, >10, >15, >30). Final report may be based on results from a scale that best captures the variability in real data. Down-sampling of reads available may be explored as necessary.

Marker-Level Evaluation:

At each marker, fit mixed effect model (Appendix) that evaluates marker specific sample effect, experiment run and reagent lot effects. Report the statistics summarized in the Appendix, which include SDs correspond to (1) intra-run/lot variability (2) variability due to run/lot effect and (3) total variability across run/lot and technical replicates excluding sample effect (i.e., interrun/lot variability). Statistics testing is also reported for the significance of between run/lot effect relative to within run/lot effect. If p-value is too small (exact threshold is to be determined), then marker is flagged as undesirable.

Panel-Level Evaluation:

Principal component analysis can be done to visually evaluate the magnitude of intra- and inter-run/lot variability with respect to the total variability across all 24 biological samples. Results generated from marker-level evaluation (6.3.4.2) are summarized across makers. Summary includes but not limited to:
Visualizing each statistic across all markers
Computing average and SD using statistics from all markers

Call Concordance Evaluation:

Compute ‘no-call’ rate per assay using a set of markers of interest. Fit a mixed effect model using ‘no-call’ rate and report results. Determine whether run/lot effect is significantly high. Also record overall ‘no-call’ rate for future reference. As variant occurrence in the 851 makers is very low (e.g., one sample may carry at most one or two variants, with the exception of UHR and other pooled samples), statistical evaluation examining experimental run and reagent lot effects can be focused on a few markers and/or a few samples with variant calls. Call outcomes across runs, lots, and technical replicates are evaluated descriptively at a few markers of interest. Outcomes are tabularized by call status (no-call, reference, variant), by run/lot and by sample.

Fusion Calling:

If necessary, the fusion calls from a primary analysis are to be further processed to meet final fusion calling criteria. At each marker, outcome from each assay is either positive or not-detected. Evaluation is focused on a pre-specified 146 fusion panel. In the future and if time allows, similar analysis may be extended to a larger panel, such as markers with known fusions in the control samples.

Call Concordance Evaluation:

As fusion occurrence in the 146 makers is very low (e.g., one sample may carry at most one or two fusions), statistical evaluation examining experimental run and reagent lot effects can be focused on a few markers and/or a few samples with positive calls. Fusion call outcomes across runs, lots, and technical replicates is evaluated descriptively at a few markers of interest. Outcomes are tabularized by call status (not-detected or positive), by run/lot and by sample.

Gene Expression Analysis:

The analysis is done on normalized count expression data to make them comparable across samples. The principal component analysis is used to evaluate visually overall consistency in expression level measurement within and between run/lots for each cohort of samples. For each gene, fit a mixed effect model using normalized counts and report results. Genes with significant intra- or inter-run/lot effect (threshold to be determined later) is included in the black list. Assessment of mRNA Integrity: Genome-wide and gene-specific mRNA integrity are assessed analytically using mRIN statistic. mRIN is defined as the negative average of modified Kolmogorov-Smirnov (KS) statistics that quantifies the 3′ bias and alteration in gene expression.

$$mRIN = - \frac{1}{N} {\sum }_{g = 1}^{N} {mKS}_{g}$$

where mKS_{g} are median-centred KS_{g} across all samples for each gene. For gene-specific degradation, the correlation between mKS_{g} and mRIN are calculated. RNA integrity is also assessed at the transcript level using TIN, an algorithm of RSeQC testing entropy level with scores ranging from 0 to 100². The median TIN score across all the transcripts can be used to measure the RNA integrity at the sample level and is compared to mRIN.

### Acceptance Criteria

For an RNASeq-based commercial product which focuses on gene expression levels and variant calling, the variant calls are dependent on the presence or absence of reads, and therefore cannot be corrected algorithmically. For an RNASeq-based commercial product, this study provides the initial data to evaluate reagent effects to the classifiers as these are developed in the future.

Mixed Effect Modeling:

For simplicity, denote the four experiment runs as Run 1, 2, 3, and 4, and the three reagent lots as Lot 1, 2, and 3. To explicitly model run/lot effect, assume that Run 4 (Experiment 2 plate 3) uses Lot 2. If Run 4 uses Lot 3 in real experiment, then the effect of Lot 2 and Lot 3 is exchanged.

Only a limited combination of Run and Lot exists: Lot1-Run1, Lot1-Run2, Lot2-Run3, Lot2-Run4, Lot3-Run3. In particular, Lot 1 is confounded with Run 1 and Run2 as a joint, thus run and lot effects cannot be completely separable. Run/Lot effect as a whole is modeled, then descriptively explore the difference in a subset of runs and a subset of lots if time allows.

Given a response value Y from each assay, fit mixed effect model: Y˜Sample+Run/Lot+Error where ‘Sample’ is the fixed main effect of sample, ‘Run/Lot’ is the main effect of run/lot and modeled as a random effect with i.i.d. Normal (0, σ²ᵣₗ), and ‘Error’ is a random error term accounting for technical replicates within Run/Lot and modeled as i.i.d. Normal (0, σ²_{ε}). Run/Lot and Error terms are pair-wise independent. In this model, σ_{ε} corresponds to intra-run/lot SD, σ²ᵣₗ corresponds to between run/lot SD. Total variability across lots, runs, and technical replicates (inter-run/lot variability) can be computed by aggregating intra-run/lot variability and between run/lot variability. After fitting the model, report (1) estimate of intra-run/lot SD, (2) estimate of run/lot SD, (3) estimate of inter-run/lot SD, and (4) a statistic testing for significance of run/lot effect.

### Example 2

A robust pipeline for capturing transcriptional data, mutations, variants and fusions all from the same RNA sample was developed and tested in a feasibility study to determine the outline of adding richer genomic content to train a genomic classifier to improve the specificity of diagnosing benign nodules while maintaining high sensitivity.

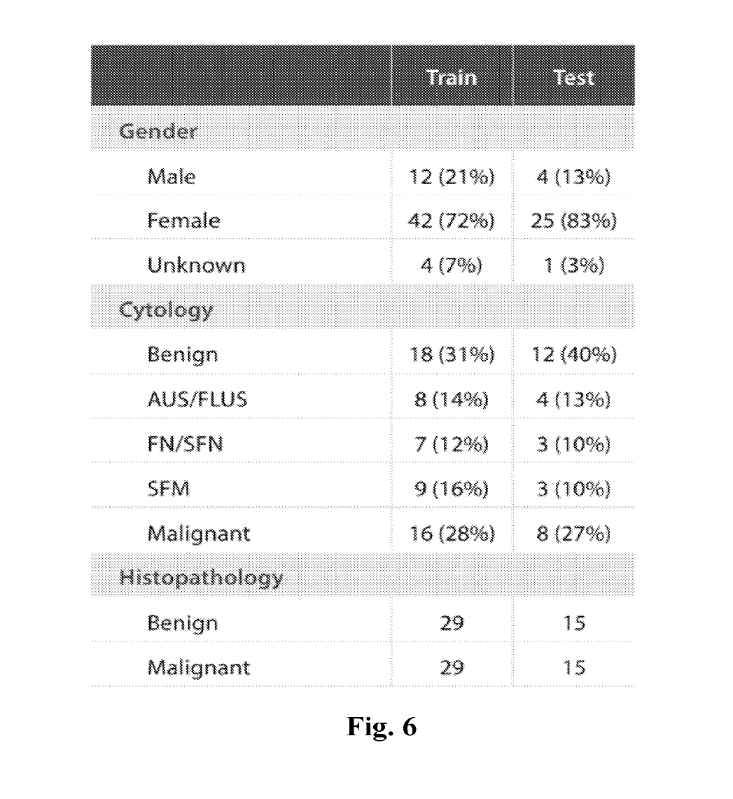

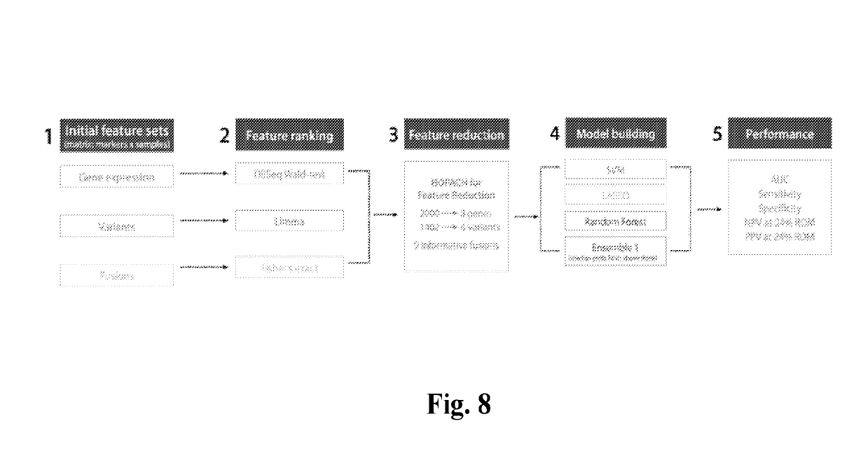

FNA biopsy samples from 88 patients were collected preoperatively and nucleic acids were isolated. The patients underwent thyroidectomies and the surgical tissue was diagnosed by a panel of histopathology experts. The cohort was balanced with 44 malignant (PTC, HCC, FC, MTC, and WDC-NOS) and 44 benign nodules (BFN, FA, HCA, LCT, NHP, and HTA). Training (n=58) and testing (n=30) sets were defined by carefully balancing cytology and histology, and classifier training was conducted in a blinded manner (FIG. 6). Samples were subjected to NGS with 15 nanograms (ng) of RNA input. Classification models were evaluated within the training set in crossvalidation according to overall performance (FIG. 8). The best model was then selected to analyze the test set.

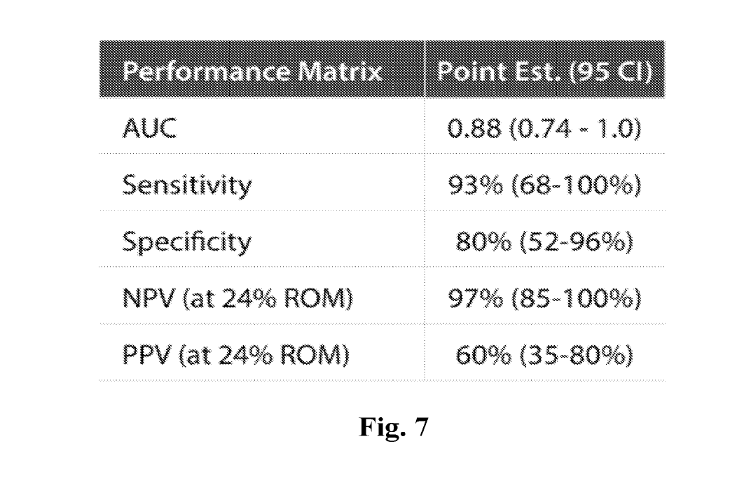

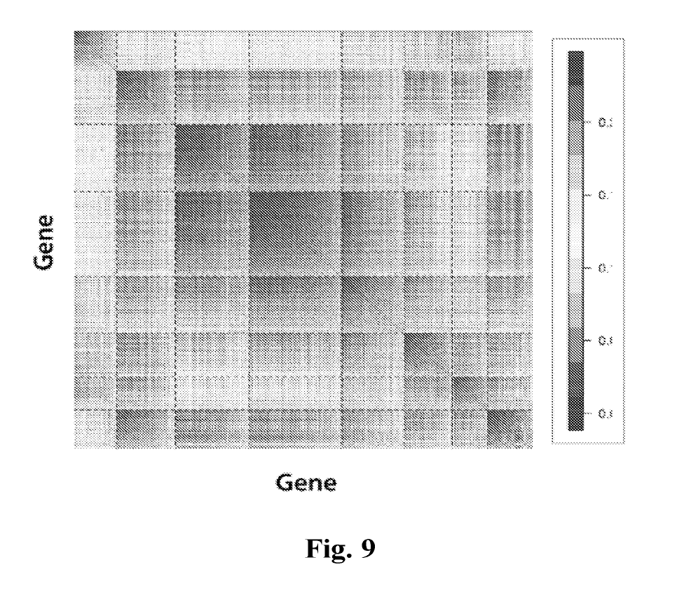

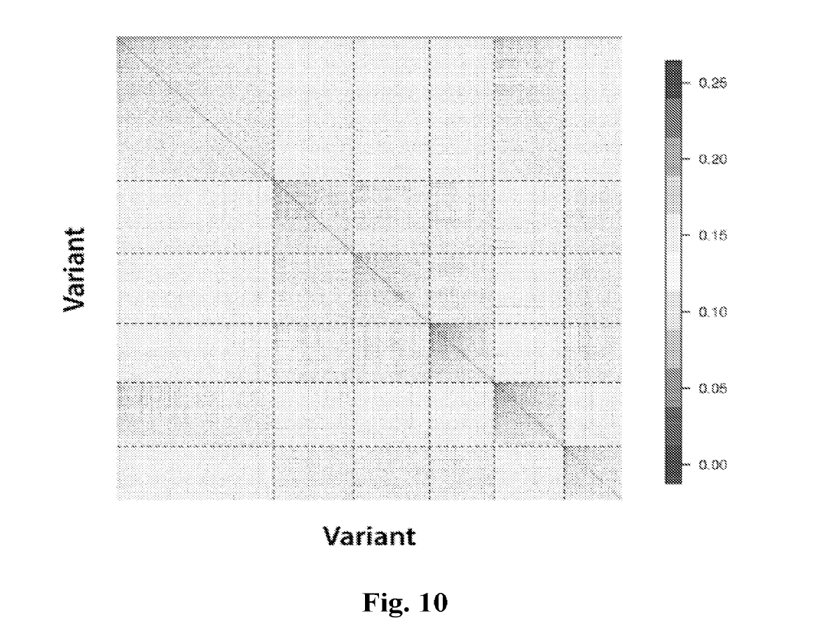

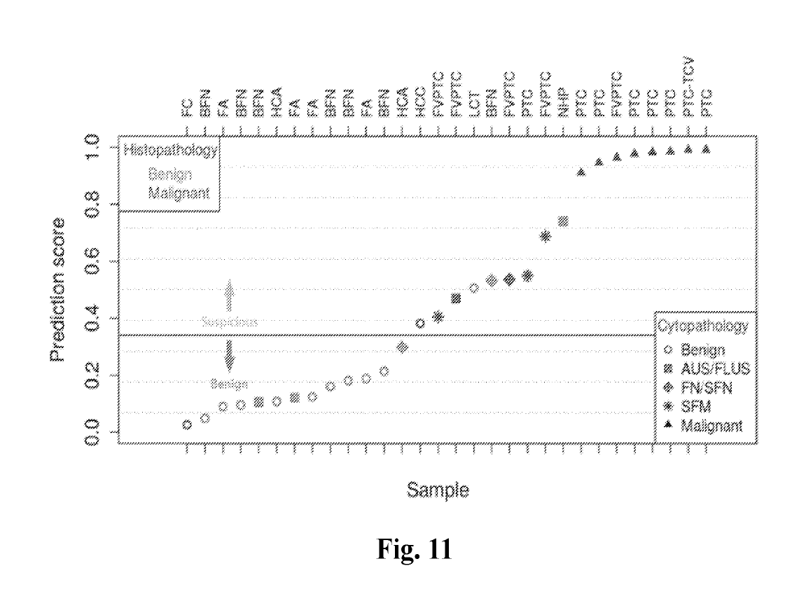

The top 2000 differentially expressed genes, 1402 sequence variants and 9 fusion-pairs were used to develop several models (FIG. 9 and FIG. 10). The best model uses an Ensemble score (median probability) from three models (SVM, LASSO, Random Forest). In the test set, this classifier yielded an overall AUC of 0.88, with a sensitivity and specificity of 93% and 80% (FIG. 7, FIG. 11).

As shown in FIG. 9, the top 2000 genes were selected with the most significant p-values derived from DEseq negative binomial Wald-test. All of them have adjusted p-value <0.05. Hierarchical Ordered Partitioning And Collapsing Hybrid (HOPACH) cluster is built using 1—Pearson correlation as pair-wise distance, where the correlation is calculated on normalized expression counts across all samples between each pair of genes. The visualization displays genes reordered based on their correlation to each other and there are 8 distinct clusters where genes within each cluster are more correlated to each other than genes outside the cluster. To avoid potential collinearity and model instability, one representative gene is selected from each of the eight clusters to be fed into the downstream classification engine. When clustering is used, the highest ranking gene from each cluster can be used for model building.

As shown in FIG. 10, the top 1402 variants were selected with the most significant p-values derived from Limma test. All of them have p-value <0.05. HOPACH cluster is built using 1—Pearson correlation as pair-wise distance, where the correlation is calculated on variant allele frequency across all samples between each pair of variants. The visualization displays variants reordered based on their correlation to each other and there are 6 distinct clusters where variants within each cluster are more correlated to each other than variants outside the cluster. To avoid potential colinearity and model instability, one representative variant is selected from each of the six clusters to be fed into the downstream classification engine.

As shown in FIG. 11, each point represents one of the 30 validation samples. They are ordered by their classification prediction score (ensemble score) from small to large. The color of the point indicates the true histopathology status of the sample: dark gray is indicative of malignant and light gray is indicative of benign. The labels provided on the top of the figure are the histopathology subtype of each sample. The shape of the point indicates the cytology Bethesda category of each sample. For example, a circle shape is indicative of benign and a triangle shape is indicative of malignant. The gray horizontal line is the cut-off of the final classifier where samples above the line are classified as suspicious and samples below the line are classified as benign.

Classifiers with high sensitivity and improved specificity can be developed from a combination of features generated using our NGS assay. This feasibility study demonstrates the principle of how counts, variants and fusions can be effectively combined.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-80. (canceled)

81. A method for processing a biological sample, comprising:
(a) assaying one or more nucleic acid sequences from said biological sample to obtain a biological dataset comprising gene expression levels, sequence variant information, or a combination thereof corresponding to said one or more nucleic acid sequences;
(b) comparing said biological dataset assayed in (a) to a second dataset comprising gene expression levels, sequence variant information, or a combination thereof corresponding to one or more nucleic acid sequences of a control sample;
(c) assigning a call to said one or more nucleic acid sequences of said biological dataset based on said comparing of (b), wherein said call is a no-call, a reference-call, or a noise-call;
(d) assigning said noise-call to a nucleic acid sequence of said biological dataset; and
(e) upon assigning said noise-call to said nucleic acid sequence, (i) flagging said nucleic acid sequence within said biological dataset, or (ii) removing said nucleic acid sequence from said biological dataset, to produce said modified biological dataset.

82. The method of claim 81, wherein said biological dataset comprises said gene expression levels.

83. The method of claim 81, wherein said biological dataset comprises said sequence variant information.

84. The method of claim 81, wherein said second dataset comprises said gene expression levels.

85. The method of claim 81, wherein said second dataset comprises said sequence variant information.

86. The method of claim 81, wherein said flagging comprises weighting said nucleic acid sequence differently from nucleic acid sequences of said biological dataset that are not assigned said noise-call.

87. The method of claim 81, wherein said assaying comprises assaying a first portion of said biological sample separately from assaying a second portion of said biological sample.

88. The method of claim 81, wherein said biological sample is obtained from a first source and a second source, wherein said first source and said second source are different.

89. The method of claim 81, wherein said comparing further comprises determining a difference in expression level between said gene expression levels of said one or more nucleic acid sequences from said biological sample compared to said gene expression levels of one or more nucleic acid sequences of said control sample having at least about 90% homology to said one or more nucleic acid sequences from said biological sample.

90. The method of claim 81, wherein said comparing further comprises determining a presence or an absence of a fusion in said one or more nucleic acid sequences of said biological sample compared to a presence or an absence of said fusion in one or more nucleic acid sequences of said control sample having at least about 90% homology to said one or more nucleic acid sequences of said biological sample.

91. The method of claim 81, wherein said comparing further comprises determining a presence or an absence of a sequence variant, a sequence variant count number, or a combination thereof in said one or more nucleic acid sequences of said biological sample compared to a present or an absence of said sequence variant, said sequence variant count number, or a combination thereof in one or more nucleic acid sequences of said control sample having at least about 90% homology to said one or more nucleic acid sequences of said biological sample.

92. The method of claim 81, wherein said biological sample is independent from said control sample.

93. The method of claim 81, wherein nucleic sequence assigned said noise-call in (d) comprises a transcript degradation, an impartial fragmentation, an incomplete library preparation, a 3′ to 5′ bias, a polymerase processivity, a polymerase sequence bias, or any combination thereof.

94. The method of claim 81, wherein said modified biological dataset comprises one or more nucleic acid sequences assigned a no-call, a reference call, or a combination thereof.

95. The method of claim 81, wherein at least about 70% of the one or more nucleic acid sequences of said biological sample have at least about 90% sequence homology to a nucleic acid sequence of said one or more nucleic acid sequences of said control sample.

96. The method of claim 81, wherein said nucleic acid sequence assigned said noise-call in (d) comprises at least about 90% sequence homology to BRAF, HRAS, KRAS, NRAS, TSHR, or RET, or any fragment thereof.

97. The method of claim 81, wherein said nucleic acid sequence assigned said noise-call in (d) comprises at least about 90% sequence homology to TSHR, RET, NRAS, TP53, PAX8, FAT1, VT11A, BRAF, HRAS, or KRAS, or any fragment thereof.

98. The method of claim 81, further comprising, employing said modified biological dataset to train a trained algorithm.

99. The method of claim 81, wherein said biological sample is obtained from a subject having or suspected of having a thyroid or lung disease condition.

100. The method of claim 81, further comprising, prior to (a), obtaining said biological sample from said subject by fine needle aspiration.

101. The method of claim 81, wherein said biological sample is cytologically indeterminate.

102. The method of claim 81, wherein said control sample is obtained from a subject suspected of having or having been diagnosed with a disease.

103. The method of claim 81, wherein said one or more nucleic acid sequences of said control sample are associated with said noise-call.

104. The method of claim 81, further comprising modifying said biological data set by removing said nucleic acid sequence from said biological dataset.


SEQUENCE LISTING (7 sequences)

SEQ ID NO: 1 - RNA, 18 nt, Unknown
  Description of Unknown reference sequence
augucgauuguagcguaa

SEQ ID NO: 2 - RNA, 12 nt, Artificial Sequence
  Description of Artificial Sequence Synthetic oligonucleotide
auuguagcguaa

SEQ ID NO: 3 - RNA, 14 nt, Artificial Sequence
  Description of Artificial Sequence Synthetic oligonucleotide
auguuguagcguaa

SEQ ID NO: 4 - RNA, 13 nt, Artificial Sequence
  Description of Artificial Sequence Synthetic oligonucleotide
augucuagcguaa

SEQ ID NO: 5 - RNA, 14 nt, Artificial Sequence
  Description of Artificial Sequence Synthetic oligonucleotide
auauuguagcguaa

SEQ ID NO: 6 - RNA, 12 nt, Artificial Sequence
  Description of Artificial Sequence Synthetic oligonucleotide
augguagcguaa

SEQ ID NO: 7 - RNA, 16 nt, Artificial Sequence
  Description of Artificial Sequence Synthetic oligonucleotide
auggauuguagcguaa